(19) United States
(12) Patent Application Publication
(10) Pub. No.: US 2025/0061574 A1
(43) Pub. Date: Feb. 20, 2025

(54) MACHINE LEARNING ENABLED DIAGNOSIS AND LESION LOCALIZATION FOR NASCENT GEOGRAPHIC ATROPHY IN AGE-RELATED MACULAR DEGENERATION

(71) Applicant: Genentech, Inc., South San Francisco, CA (US)

(72) Inventors: Heming YAO, Ann Arbor, MI (US); Miao ZHANG, Foster City, CA (US); Seyed Mohammadmohsen HEJRATI, Stanford, CA (US)

(21) Appl. No.: 18/939,497

(22) Filed: Nov. 6, 2024

Related U.S. Application Data
(63) Continuation of application No. PCT/US2023/021420, filed on May 8, 2023.
Application No. PCT/US2023/021420 is a continuation-in-part of application No. PCT/US22/47944, filed on Oct. 26, 2022.
(60) Provisional application No. 63/339,333, filed on May 6, 2022.
(60) Provisional application No. 63/484,150, filed on Feb. 9, 2023.

(51) Int. Cl. G06T 7/00; A61B 3/00; A61B 3/10; A61B 3/12; G06T 7/73; G16H 50/20

(52) U.S. Cl. CPC G06T 7/0014; A61B 3/0025; A61B 3/0058; A61B 3/102; A61B 3/1225; G06T 7/74; G16H 50/20; G06T 2207/10101; G06T 2207/20076; G06T 2207/20081; G06T 2207/20084; G06T 2207/30041; G06T 2207/30096; G06T 2207/30204

(57) ABSTRACT

A method and system for detecting nascent geographic atrophy. An optical coherence tomography (OCT) volume image of a retina of a subject is received. Using a deep learning model, an output is generated using the OCT volume image in which the output indicates whether nascent geographic atrophy is detected. A map output is generated for the deep learning model using a saliency mapping algorithm, wherein the map output indicates a level of contribution of a set of regions in the OCT volume image to the output generated by the deep learning model.

### CROSS-REFERENCE TO RELATED APPLICATIONS

This application is a continuation of International Application No. PCT/US2023/021420 filed on May 8, 2023, which is related to and claims the benefit of the priority date of U.S. Provisional Application 63/339,333, filed May 6, 2022, entitled “Machine Learning Enabled Diagnosis and Lesion Localization for Nascent Geographic Atrophy in Age-Related Macular Degeneration” and U.S. Provisional Application No. 63/484,150, filed Feb. 9, 2023, entitled “Machine Learning Enabled Diagnosis and Lesion Localization for Nascent Geographic Atrophy in Age-Related Macular Degeneration,” and is a continuation-in-part of International Application PCT/US2022/047944, filed Oct. 26, 2022, entitled “Methods and Systems for Biomarker Identification and Discovery,” each of which is incorporated herein by reference in its entirety.

### TECHNICAL FIELD

The subject matter described herein relates generally to machine learning and more specifically to machine learning based diagnosis and lesion localization techniques for nascent geographic atrophy (nGA) in age-related macular degeneration (AMD).

### BACKGROUND

Various imaging techniques have been developed to capture medical images of tissues, which may then be analyzed to determine the presence or progression of diseases. For example, optical coherence tomography (OCT) refers to a technique where light waves are used to capture two-dimensional slice images and three-dimensional volume images of tissues such as retinas of patients, which may then be analyzed to diagnose, monitor, treat, etc., the patients. However, the analyses of such images, which may include a large amount of data, are performed manually, and usually by subject matter experts, and as such can be cumbersome and very expensive. Thus, it may be desirable to have methods and systems that facilitate the consistent, accurate, and quick analyses of large amounts of medical images such as OCT images for use in the diagnosis, monitoring and treatment of patients.

### SUMMARY

The following summarizes some embodiments of the present disclosure to provide a basic understanding of the discussed technology. This summary is not an extensive overview of all contemplated features of the disclosure and is intended neither to identify key or critical elements of all embodiments of the disclosure nor to delineate the scope of any or all embodiments of the disclosure. Its sole purpose is to present some concepts of one or more embodiments of the disclosure in summary form as a prelude to the more detailed description that is presented later.

In one or more embodiments, a system comprises at least one data processor; and at least one memory storing instructions, which when executed by the at least one data processor, result in operations comprising: applying a machine learning model trained to determine, based at least on an optical coherence tomography (OCT) volume of a patient, a diagnosis of nascent geographic atrophy (nGA) for the patient; determining, based at least on a saliency map associated with the diagnosis of nascent geographic atrophy, a location of one or more nascent geographic atrophy lesions; and verifying the diagnosis of nascent geographic atrophy and/or the location of the one or more nascent geographic atrophy lesions.

In one or more embodiments, a computer-implemented method is provided. The method includes applying a machine learning model trained to determine, based at least on an optical coherence tomography (OCT) volume of a patient, a diagnosis of nascent geographic atrophy (nGA) for the patient; determining, based at least on a saliency map associated with the diagnosis of nascent geographic atrophy, a location of one or more nascent geographic atrophy lesions; and verifying the diagnosis of nascent geographic atrophy and/or the location of the one or more nascent geographic atrophy lesions.

In one or more embodiments, a non-transitory computer readable medium stores instructions, which when executed by at least one data processor, result in operations comprising: applying a machine learning model trained to determine, based at least on an optical coherence tomography (OCT) volume of a patient, a diagnosis of nascent geographic atrophy (nGA) for the patient; determining, based at least on a saliency map associated with the diagnosis of nascent geographic atrophy, a location of one or more nascent geographic atrophy lesions; and verifying the diagnosis of nascent geographic atrophy and/or the location of the one or more nascent geographic atrophy lesions.

In one or more embodiments, a method is provided. The method includes receiving an optical coherence tomography (OCT) volume image of a retina of a subject; generating, via a deep learning model, an output using the OCT volume image in which the output indicates whether nascent geographic atrophy is detected; and generating a map output for the deep learning model using a saliency mapping algorithm, wherein the map output indicates a level of contribution of a set of regions in the OCT volume image to the output generated by the deep learning model.

In one or more embodiments, a system comprises a non-transitory memory; and a hardware processor coupled with the non-transitory memory and configured to read instructions from the non-transitory memory to cause the system to: receive an optical coherence tomography (OCT) volume image of a retina of a subject; generate, via a deep learning model, an output using the OCT volume image in which the output indicates whether nascent geographic atrophy is detected; generate a map output for the deep learning model using a saliency mapping algorithm, wherein the map output indicates a level of contribution of a set of regions in the OCT volume image to the output generated by the deep learning model; and display the map output.

In one or more embodiments, a system comprises a non-transitory memory; and a hardware processor coupled with the non-transitory memory and configured to read instructions from the non-transitory memory to cause the system to: train a deep learning model using a training dataset that includes training OCT images that have been labeled as evidencing nascent geographic atrophy or not evidencing nascent geographic atrophy to form a trained deep learning model; receive an optical coherence tomography (OCT) volume image of a retina of a subject; generate, via the trained deep learning model, a classification score using the OCT volume image in which the classification score indicates whether nascent geographic atrophy is detected; generate a saliency volume map for the OCT volume image using a saliency mapping algorithm, wherein the saliency volume map indicates a level of contribution of a set of regions in the OCT volume image to the diagnosis of geographic atrophy generated by the deep learning model; detect a set of potential biomarker regions in the OCT volume image using the saliency volume map; and generate a report that confirms that nascent geographic atrophy is detected when at least one potential biomarker region of the set of potential biomarker regions meets a set of criteria and when the classification score meets a threshold.

### DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 is a block diagram of a networked system 100 in accordance with one or more example embodiments.

FIG. 2 is a flowchart of a process for processing an OCT volume image of a retina of a subject to determine whether the OCT volume image evidences a selected health status category for the retina in accordance with one or more example embodiments.

FIG. 3 is a flowchart of a process for identifying biomarkers in an OCT volume image of a retina of a subject in accordance with one or more example embodiments.

FIG. 4A is a flowchart of a process 400 for artificial intelligence assisted nascent geographic atrophy (nGA) detection in accordance with one or more example embodiments.

FIG. 4B is a flowchart of a process for processing an OCT volume image of a retina of a subject to determine whether the OCT volume image evidences nascent geographic atrophy (nGA) in accordance with one or more example embodiments.

FIG. 5 illustrates an annotated OCT slice image and a corresponding heatmap for the annotated OCT slice image in accordance with one or more example embodiments.

FIG. 6 is an illustration of different maps in accordance with one or more example embodiments.

FIG. 7 depicts a system diagram illustrating an example of a nascent geographic atrophy detection system, in accordance with some example embodiments.

FIG. 8A is an illustration of one example of a model for processing a 3D OCT volume in accordance with one or more example embodiments.

FIG. 8B illustrates one example of an implementation for a classifier 802 that may be used to implement a classifier in accordance with one or more example embodiments.

FIG. 9 depicts an exemplary data flow diagram with data split statistics in accordance with one or more example embodiments.

FIG. 10 is an illustration of an output workflow for outputs generated from an OCT volume in accordance with one or more example embodiments.

FIG. 11A is an illustration of a confusion matrix 1100 in accordance with one or more example embodiments.

FIG. 11B is a graph of statistics for a 5-fold cross-validation in accordance with one or more example embodiments.

FIG. 12A is an illustration of OCT images 1200 (e.g. B-scans) in which nGA lesions have been detected in accordance with one or more example embodiments.

FIG. 12B is a graph 1202 of the precision-recall curves for a 5-fold cross validation in accordance with one or more example embodiments.

FIG. 12C is illustration of a confusion matrix 1204 in accordance with one or more example embodiments.

FIG. 13 is an illustration of OCT images 1300 that have been annotated with bounding boxes in accordance with one or more example embodiments.

FIG. 14 illustrates an example neural network that can be used to implement a computer-based model according to various embodiments of the present disclosure.

FIG. 15 depicts a block diagram illustrating an example of a computing system, in accordance with some example embodiments.

When practical, similar reference numbers denote similar structures, features, or elements.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Overview

Medical imaging technologies are powerful tools that can be used to produce medical images that allow healthcare practitioners to better visualize and understand the medical issues of their patients, and as such provide the same more accurate diagnoses and treatment options. For example, optical coherence tomography (OCT) is a noninvasive imaging technique that is particularly popular for capturing images of the retina. OCT may be described as an ultrasonic scanning technique that scatters light waves from tissues to generate OCT images in the form of two-dimensional (2D) images and/or three-dimensional (3D) images of the tissues, similar to ultrasound scans that use sound waves to scan tissues. A 2D OCT image may also be referred to as an OCT slice, OCT cross-sectional image, or OCT scan (e.g., OCT B-scan). A 3D OCT image may be referred to as an OCT volume image and may be comprised of many OCT slice images. OCT images may then be used for the diagnosis, monitoring and/or treatment of patients from whom the images are obtained. For example, OCT slice images and OCT volume images of the retinas of a patient with age-related macular degeneration (AMD) may be analyzed to provide AMD diagnoses and treatment options to the patient.

Although OCT images of retinas may contain valuable information about patients' ophthalmological conditions, extracting the information from the OCT images can be a resource-intensive and difficult task, leading to erroneous conclusions to be drawn about the information contained in the OCT images. For example, when treating a patient with an eye disease such as AMD, a large set of OCT slices of the retinas of the patient may be obtained, and a set of trained human reviewers may be tasked with manually identifying biomarkers of AMD in the set of OCT slices. Such a process, however, can be cumbersome and challenging, leading to slow, inaccurate, and/or variable identification of biomarkers of retina diseases. Although subject matter experts who are trained at reviewing OCT images may be used to improve the accuracy of biomarker identifications, the process may still be laborious, may have inherent undesirable variability between reviewers, and may be particularly costly. Accordingly, relying on such subject matter experts to review such large sets of OCT slices may not provide health care providers with the efficient, cost-effective, consistent, and accurate mechanism desired for identifying biomarkers of diseases such as AMD. Further, manual review of OCT images may be even less successful at discovering new biomarkers that are prognostic of the future development of retina diseases.

For example, geographic atrophy (GA) may be a late-stage, vision-threatening complication of AMD. While color fundus photography (CFP) or fundus autofluorescence (FAF) can be used to identify GA, there may already be substantial loss of outer retinal tissue by the time a subject matter expert is able to see evidence of GA on these types of images. Determining ways in which to slow or prevent the onset of GA in the early stages of AMD may benefit from identifying early signs or predictors of GA onset. For example, biomarkers for early identification and/or prediction of GA onset can be used to identify high-risk individuals to enrich clinical trial populations, serve as biomarkers for different stages of AMD progression, and/or potentially act as an earlier endpoint in clinical trials aiming to prevent the onset of GA.

OCT images have been used to identify nascent geographic atrophy (nascent GA or nGA), which may be a strong predictor that the onset of GA is near (e.g., within 6-30 months). Identifying optical coherence tomography (OCT) signs of nascent geographic atrophy (nGA) associated with geographic atrophy onset can help enrich trial inclusion criteria. For example, in some cases, retinas that show nascent GA in OCT images have greater than a 70-fold increased risk of developing GA as compared to those retinas that do not show nascent GA. Thus, nascent GA may be prognostic indicator of a progression from early AMD to GA. Examples of the anatomic biomarkers that define nascent GA in OCT images include, but are not limited to, subsidence of the inner nuclear layer (INL) and outer plexiform layer (OPL), hyporeflective wedge-shaped bands within Henle's fiber layer, or both.

The accurate identification of nascent GA could improve the feasibility of evaluating preventative treatments for the onset of GA. The manual grading of all B-scans in optical coherence tomography volumes can be a laborious and operationally expensive undertaking, especially as B-scan densities increase to improve coverage of the macula. Increasing the speed and decreasing the computational cost of the process would improve the utility of optical coherence tomography biomarkers or endpoints.

Thus, the embodiments described herein provide artificial intelligence (AI)-based systems and methods for quickly, efficiently, and accurately detecting whether an OCT volume image of a retina evidences a selected health status category for the retina. The selected health status category may be, for example, a retinal disease (e.g., AMD) or a stage of retinal disease. In one or more embodiments, the selected health status category is nascent GA. In other embodiments, the selected health status category may be another stage of AMD progression (e.g., early AMD, intermediate AMD, GA, etc.). A deep learning model may be trained to receive an OCT volume image and generate a health indication output that indicates whether the OCT volume image evidences a selected health status category (e.g., nascent GA) for the retina. For example, the health indication output may indicate a level of association between the OCT volume image and the selected health status category. This level of association may be no association, some association, or a full association. The deep learning model may include, for example, a neural network model. As one non-limiting example, the deep learning model may generate a health indication output that is a probability (e.g., between 0.00 and 1.00) that indicates the level of association between the OCT volume image and the selected health status category.

Further, the systems and methods described herein may be used to quickly, efficiently, and accurately identify biomarkers of retina diseases and/or prognostic biomarkers of future retinal disease developments. For example, the systems and methods described herein may be used to identify a set of biomarkers in an OCT volume image that indicate or otherwise correspond to the selected health status category. The systems and methods may also be used to identify a set of prognostic biomarkers in the OCT volume image that are prognostic for the selected health status category (e.g., a progression to the selected health status category within a selected period of time).

In one or more embodiments, a health status identification system that includes a deep learning model is used to process OCT volume images. The health identification system uses the deep learning model, which may include a neural network model, to generate a health indication output that indicates whether an OCT volume image evidences a selected health status category. In some instances, the selected health status category may be one out of a group of health status categories of interest. In one or more embodiments, the selected health status category is a selected stage of AMD. The selected stage of AMD may be, for example, nascent GA.

In one or more embodiments, the health status identification system uses a saliency mapping algorithm (also referred to as a saliency mapping technique) to generate a map output for the deep learning model that indicates whether a set of regions in the OCT volume image is associated with the selected health status category. The saliency mapping algorithm may be used to identify a level of contribution (or a degree of importance) of various portions of the OCT volume image to the health indication output generated by the deep learning model for the given OCT volume image. The health status identification system may use the map output to identify biomarkers in the OCT volume image. A biomarker may indicate that the OCT volume image currently evidences the selected health status category for the retina. In some instances, a biomarker may be prognostic in that it indicates that the OCT volume image is prognostic for the retina progressing to the selected health tatus category within a selected period of time (e.g., 6 months, 1 year, 2 years, 3 years, etc.).

The saliency mapping algorithm described above may be implemented in various ways. One example of a saliency mapping algorithm is gradient-weighted Class Activation Mapping (Grad-CAM), a technique that provides “visual explanations” in the form of heatmaps for the decisions that a deep learning model makes when performing predictions. That is, Grad-CAM may be implemented for a trained deep learning model to generate saliency maps or heatmaps of OCT slice images in which the heatmaps indicate (e.g., using colors, outlines, annotations, etc.) the regions or locations of the OCT slice images that the neural network model uses in making determinations and/or predictions about stages of disease for the retinas shown in the OCT slice images. In one or more embodiments, Grad-CAM may determine the degree of importance of each pixel in an OCT slice image to the health indication output generated by the deep learning model. Additional details about Grad-CAM may be found in R. R. Selvaraju et al., “Grad-CAM: Visual Explanations from Deep Networks via Gradient-based Localization,” Arxiv: 1610.02391 (2017), which is incorporated by reference herein in its entirety. Other non-limiting examples of saliency mapping techniques include class activation mappings (CAMs), SmoothGrad, the Low-Variance Gradient Estimator for Variational Inference (VarGrad), and/or the like.

The saliency map generated by the saliency mapping algorithm may then be used to localize one or more potential biomarkers on a given OCT slice image. For example, the saliency map may be used to generate a bounding box around each potential biomarker or potential biomarker region in the OCT slice image. Thus, each bounding box may localize the potential biomarker. In one or more embodiments, a scoring metric (e.g., confidence score) may be used to determine which bounding boxes are or contain one or more biomarkers for a selected health status category.

Using the health status identification system with the deep learning model and the saliency mapping algorithm to classify retinal health status and identify biomarkers for a selected health status category in an OCT volume image may reduce the time and cost associated with evaluating the retinas of subjects and may improve the efficiency and accuracy with which diagnosis, monitoring, and/or treatment can be implemented. Further, using the embodiments described herein may allow subjects to be added to clinical trials at earlier stages of their AMD progression and may improve the informative potential of such clinical trials. Still further, using the embodiments described herein may reduce the overall computing resources used and/or speed up a computer's performance with respect to classifying retinal health status, predicting future retinal health status, and/or identifying biomarkers for a selected health status category.

In some example embodiments, a deep learning model may be trained to detect nascent geographic atrophy based on optical coherence tomography imaging. The deep learning model may be trained, based on the information about the presence or absence of nascent geographic atrophy at the eye level, to effectively identify the location of these lesions. The ability to locate nascent geographic atrophy may be critical if deploying such diagnostic tools in clinical trials, diagnosis, treatment, monitoring, research, and/or the like. In some cases, the diagnostic outputs of the deep learning model may undergo further verification or justification in the clinical setting. For example, instead of and/or in addition to presenting a diagnostic result, the deep learning model may propose one or more regions with high likelihood of containing nascent geographic atrophy lesions. Accordingly, clinicians may make the final diagnosis by examining only a subset of B-scans, or even regions from the B-scans. The deep learning model in this case should have a high recall in localizing nascent geographic atrophy lesions. Its precision should be much higher than prevalence to reduce the workload of clinicians as much as possible.

### II. Example Health Status Identification

### II.A. Example System for Health Status Identification

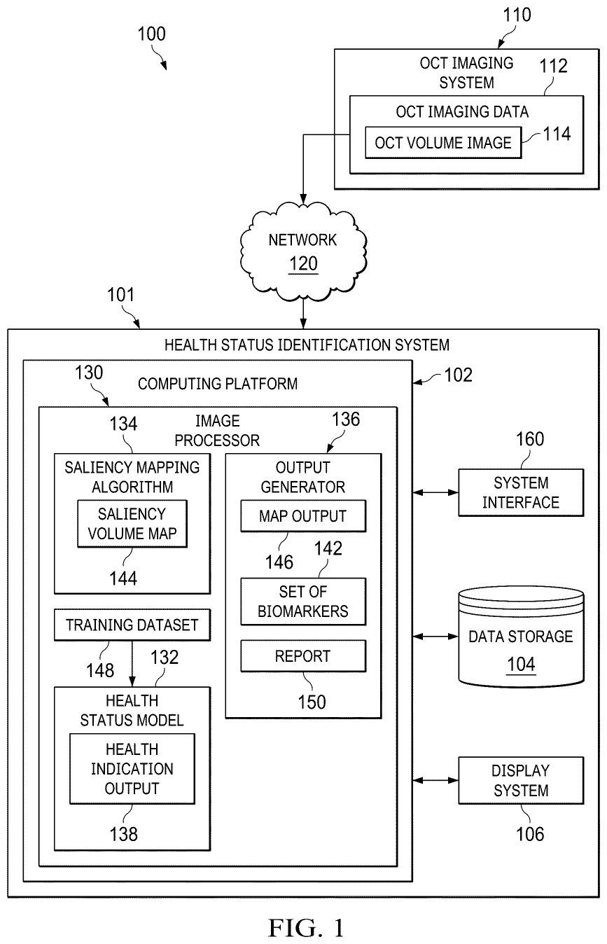

FIG. 1 is a block diagram of a networked system 100 in accordance with one or more example embodiments. Networked system 100 may include any number or combination of servers and/or software components that operate to perform various processes related to the capturing of OCT volume images of tissues such as retinas, the processing of OCT volume images via a deep learning model, the processing of OCT volume images using a saliency mapping algorithm, the identification of biomarkers that indicate current retinal health status or are prognostic of retinal health status, or a combination thereof. Exemplary servers may include, for example, stand-alone and enterprise-class servers operating a server OS such as a MICROSOFT™ OS, a UNIX™ OS, a LINUX™ OS, or other suitable server-based OS. It can be appreciated that the servers used in networked system 100 may be deployed in other ways and that the operations performed and/or the services provided by such servers may be combined or separated for a given implementation and may be performed by a greater number or fewer number of servers. One or more servers may be operated and/or maintained by the same or different entities.

The networked system 100 includes health status identification (HSI) system 101. The health status identification system 101 may be implemented using hardware, software, firmware, or a combination thereof. In one or more embodiments, the health status identification system 101 may include a computing platform 102, a data storage 104 (e.g., database, server, storage module, cloud storage, etc.), and a display system 106. Computing platform 102 may take various forms. In one or more embodiments, computing platform 102 includes a single computer (or computer system) or multiple computers in communication with each other. In other examples, computing platform 102 takes the form of a cloud computing platform, a mobile computing platform (e.g., a smartphone, a tablet, etc.), or a combination thereof.

Data storage 104 and display system 106 are each in communication with computing platform 102. In some examples, data storage 104, display system 106, or both may be considered part of or otherwise integrated with computing platform 102. Thus, in some examples, computing platform 102, data storage 104, and display system 106 may be separate components in communication with each other, but in other examples, some combination of these components may be integrated together.

The networked system 100 may further include OCT imaging system 110, which may also be referred to an OCT scanner. OCT imaging system 110 may generate OCT imaging data 112. OCT imaging data 112 may include OCT volume images (i.e., 3D OCT images) and/or OCT slice images (i.e., 2D OCT images). For example, OCT imaging data 112 may include OCT volume image 114. The OCT volume image 114 may be comprised of a plurality (e.g., 10s, 100s, 1000s, etc.) of OCT slice images. An OCT slice image may also be referred to as an OCT B-scan or a cross-sectional OCT image.

In one or more embodiments, the OCT imaging system 110 includes an optical coherence tomography (OCT) system (e.g., OCT scanner or machine) that is configured to generate OCT imaging data 112 for the tissue of a patient. For example, OCT imaging system 110 may be used to generate OCT imaging data 112 for the retina of a patient. In some instances, the OCT system can be a large tabletop configuration used in clinical settings, a portable or handheld dedicated system, or a “smart” OCT system incorporated into user personal devices such as smartphones. The OCT imaging system 110 may include an image denoiser that is configured to remove noise and other artifacts from a raw OCT volume image to generate the OCT volume image 114.

The health status identification system 101 may be in communication with OCT imaging system 110 via network 120. Network 120 may be implemented using a single network or multiple networks in combination. Network 120 may be implemented using any number of wired communications links, wireless communications links, optical communications links, or combination thereof. For example, in various embodiments, network 120 may include the Internet or one or more intranets, landline networks, wireless networks, and/or other appropriate types of networks. In another example, the network 120 may comprise a wireless telecommunications network (e.g., cellular phone network) adapted to communicate with other communication networks, such as the Internet.

The OCT imaging system 110 and health status identification system 101 may each include one or more electronic processors, electronic memories, and other appropriate electronic components for executing instructions such as program code and/or data stored on one or more computer readable mediums to implement the various applications, data, and steps described herein. For example, such instructions may be stored in one or more computer readable media such as memories or data storage devices (e.g., data storage 104) internal and/or external to various components of networked system 100, and/or accessible over network 120. Although only one of each of OCT imaging system 110 and the health status identification system 101 is shown, there can be more than one of each in other embodiments.

In some embodiments, the OCT imaging system 110 may be maintained by an entity that is tasked with obtaining OCT imaging data 112 for tissue samples of subjects for the purposes of diagnosis, monitoring, treatment, research, clinical trials, and/or the like. For example, the entity can be a health care provider (e.g., ophthalmology healthcare provider) that seeks to obtain OCT imaging data for a retina of a patient for use in diagnosing eye conditions or diseases (e.g., AMD) the patient may have. As another example, the entity can be an administrator of a clinical trial that is tasked with collecting OCT imaging data for retinas of subjects to monitor changes to the retinas as a result of the progression/regression of diseases affecting the retinas and/or effects of drugs administered to the subjects to treat the diseases. It is to be noted that the above examples are non-limiting and that the OCT imaging system 110 may be maintained by other entities and/or professionals that can use the OCT imaging system 110 to obtain OCT imaging data of retinas for the aforementioned or any other medical purposes.

In some embodiments, the health status identification system 101 may be maintained by an entity that is tasked with identifying or discovering biomarkers of tissue diseases or conditions from OCT images of the same. For example, the health status identification system 101 may be maintained by an ophthalmology healthcare provider, researcher, clinical trial administrator, etc., that is tasked with identifying or discovering biomarkers of retina diseases such as AMD. Although FIG. 1 shows the OCT imaging system 110 and the health status identification system 101 as two separate components, in some embodiments, the OCT imaging system 110 and the health status identification system 101 may be parts of the same system or module (e.g., and maintained by the same entity such as a health care provider or clinical trial administrator).

The health status identification system 101 may include an image processor 130 that is configured to receive OCT imaging data 112 from the OCT imaging system 110. The image processor 130 may be implemented using hardware, firmware, software, or a combination thereof. In one or more embodiments, the image processor 130 may be implemented within computing platform 102.

The image processor 130 may include model 132 (which may also be referred to as health status model 132), saliency mapping algorithm 134, and output generator 136. Model 132 may include a machine learning model. For example, model 132 may include a deep learning model. In one or more embodiments, the deep learning model includes a neural network model that comprises one or more neural networks. Model 132 can be used to identify (or classify) the current and/or future health status for the retina of a subject.

For example, model 132 may receive OCT imaging data 112 as input. In particular, model 132 may receive OCT volume image 114 of the retina of a subject. Model 132 may process OCT volume image 114 by processing at least a portion of the OCT slice images that make up OCT volume image 114. In some embodiments, model 132 processes every OCT slice image that makes up OCT volume image 114. Model 132 generates health indication output 138 based on OCT volume image 114 in which health indication output 138 indicates whether OCT volume image 114 evidences selected health status category 140 for the retina of the subject.

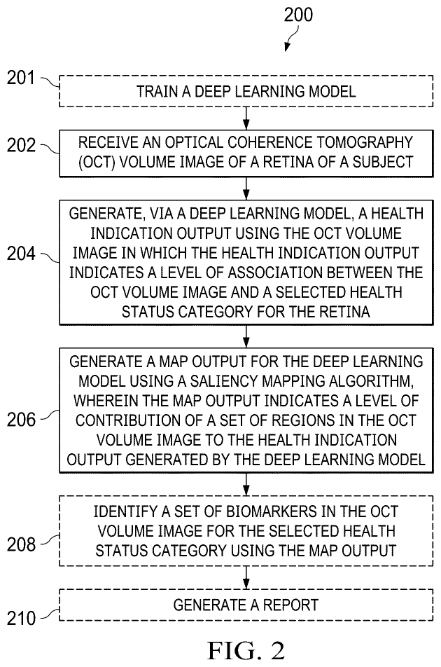

For example, the health indication output 138 may indicate a level of association between the OCT volume image 114 and selected health status category 140. This level of association may be indicated via a probability. For example, in one or more embodiments, the health indication output 138 may be a probability that indicates the level of association between the OCT volume image 114 and selected health status category 140 or how likely it is that the OCT volume image 114 evidences the selected health status category 140. This level of association may be, for example, no association (e.g., 0.0 probability), a weak association (e.g., between 0.01 and 0.4 probability), a moderate association (e.g., between 0.4 and 0.6 probability), a strong association (e.g., between 0.6 and 1.0 probability), or some other type of association. These percentages are merely some examples of probability ranges and levels of association. Other levels of association and/or other percentage ranges may be used in other embodiments. The process by which model 132 generates health indication output 138 is described in greater detail with respect to FIG. 2 below.

Selected health status category 140 may be a health status for the retina that refers to a current point in time or a future point in time (e.g., 6 months, 1 year, 2 years, etc. into the future). In other words, selected health status category 140 may represent a current health status or a future health status. The current point in time may be, for example, the time at which the OCT volume image 114 was generated within a selected interval (e.g., 1 week, 2 weeks, 1 month, 2 months, etc.) of the time at which the OCT volume image 114 was generated.

In one or more embodiments, selected health status category 140 may be a selected stage of AMD. Selected health status category 140 may be, for example, without limitation, current nascent GA or future nascent GA. In some instances, selected health status category 140 represents a stage of AMD that is predicted to lead to nascent GA within a selected period of time (e.g., 6 months, 1 year, 2, years, etc.). In other instances, selected health status category 140 represents a stage of AMD that is predicted to lead to the onset of GA within a selected period of time. In still other instances, selected health status category 140 represents a stage of AMD that is predicted to lead to nascent GA within a selected period of time. In this manner, selected health status category 140 may be for a current health status of the retina or a prediction of a future health status of the retina. Other examples of health status categories include, but are not limited to, early AMD, intermediate AMD, GA, etc.

As described above, model 132 may be implemented using a neural network model. The neural network model may include any number of or combination of neural networks. A neural network may take the form of, but is not limited to, a convolutional neural network (CNN) (e.g., a U-Net), a fully convolutional network (FCN) a stacked FCN, a stacked FCN with multi-channel learning, a feedforward neural network (FNN), a recurrent neural network (RNN), a modular neural network (MNN), a residual neural network (ResNet), an ordinary differential equations neural network (neural-ODE), a Squeeze and Excitation embedded neural network, a MobileNet, or another type of neural network. In one or more embodiments, a neural network may itself be comprised of at least one of a CNN (e.g., a U-Net), a FCN, a stacked FCN, a stacked FCN with multi-channel learning, a FNN, a RNN, an MNN, a ResNet, a neural-ODE, a Squeeze and Excitation embedded neural network, a MobileNet, or another type of neural network. In one or more embodiments, the neural network model takes the form of a convolutional neural network (CNN) system that includes one or more convolutional neural networks. For example, the CNN may include a plurality of neural networks, each of which may itself be a convolutional neural network.

In one or more embodiments, the neural network model may include a set of encoders, each of which can be a single encoder or multiple encoders, and a decoder. In some embodiments, the one or more encoders and/or the decoder may be implemented via a neural network, which may, in turn, be comprised of one or more neural networks. In some instances, the decoder and the one or more encoders may be implemented using a CNN. The decoder and the one or more encoders may also be implemented as a Y-Net (Y-shaped neural network system) or a U-Net (U-shaped neural network system). Further details related to neural network are provided below with reference to FIG. 6.

The health status identification system 101 may also be used to identify (or detect) a set of biomarkers 142 for selected health status category 140. For example, the health status identification system 101 may be used to identify set of biomarkers 142 in the OCT volume image 114 that evidence selected health status category 140 for the retina of the subject. For example, when selected health status category 140 is current nascent GA, set of biomarkers 142 may include one or more anatomic biomarkers that indicate that the OCT volume image 114 currently evidences selected health status category 140 for the retina. When selected health status category 140 represents future health status (e.g., predicted to progress to nascent GA within a selected period of time), set of biomarkers 142 may be prognostic for this future health status.

The health status identification system 101 uses saliency mapping algorithm 134 to identify set of biomarkers 142. For example, saliency mapping algorithm 134 may be used to identify the portions (or regions) of the OCT volume image 114 that most impacted or contributed the most to the health indication output 138 of model 132. For example, saliency mapping algorithm 134 may indicate the degree of importance for the various portions (or regions) of the OCT volume image 114 for selected health status category 140.

Saliency mapping algorithm 134 may include, but is not limited to, Grad-CAM, CAM, SmoothGrad, VarGrad, another type of saliency mapping algorithm or technique, or a combination thereof. The saliency mapping algorithm 134 may generate saliency volume map 144, which indicates (e.g., via a heatmap) the degree of importance for the various portions (or regions) of the OCT volume image 114 with respect to selected health status category 140. In other words, saliency volume map 144 indicates the level of contribution of the various portions of the OCT volume image 114 to the health indication output 138 generated by the model 132. Saliency volume map 144 may be comprised of a plurality of saliency maps, each of which corresponds to a different one of the plurality of OCT slice images in the OCT volume image 114. Each saliency map may visually indicate (e.g., via color, highlighting, shading, pattern, outlining, text, annotations, etc.) the regions of the corresponding OCT slice image that were most impactful to model 132 for selected health status category 140.

Output generator 136 may receive and process saliency volume map 144 to generate map output 146. In one or more embodiments, map output 146 takes the form of a filtered or modified form of saliency volume map 144. In other embodiments, map output 146 takes the form of saliency volume map 144 or a modified form of saliency volume map 144 overlaid on OCT volume image 114. Similar to how saliency volume map 144 may be comprised of multiple saliency maps (two-dimensional), map output 146 may be comprised of multiple individual two-dimensional maps. These maps may be heat maps or overlays of heat maps over OCT slice images.

In one or more embodiments, a filter (e.g., threshold filter) may be applied to saliency volume map 144 to identify a subset of the saliency maps in saliency volume map 144 to be modified. The threshold filter may be set to ensure that only those saliency maps indicating a contribution of, for example, at least one region in the corresponding OCT slice image above a selected threshold are selected for the subset. This subset of saliency maps may then be modified such that the modified saliency volume map that is formed includes fewer maps than the saliency volume map 144. In this manner, when map output 146 is generated, map output 146 may be comprised of fewer maps than saliency volume map 144. In other embodiments, other types of filtering steps and/or other preprocessing steps may be performed such that map output 146 that is generated includes a fewer number of maps than the maps in saliency volume map 144.

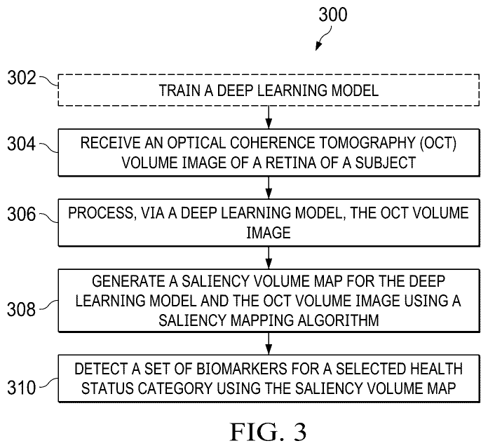

Map output 146 may indicate whether a set of regions in OCT volume image 114 is associated with the selected health status category. For example, map output 146 may indicate a level of contribution of a set of regions in OCT volume image 114 to the health indication output 138 generated by the model 132. A region may be a pixel-level region or a region formed by multiple pixels. A region may be a continuous or discontinuous region. In some embodiments, map output 146 visually localizes set of biomarkers 142. In other embodiments, map output 146 may be further processed by output generator 136 to identify which of the regions of OCT volume image 114 are or include biomarkers. The process of identifying set of biomarkers 142 using saliency mapping algorithm 134 and output generator 136 is described in greater detail with respect to FIGS. 2-3 below. In some embodiments, saliency mapping algorithm 134 is integrated with or implemented as part of output generator 136.

In some embodiments, the model 132 may be trained with training dataset 148, which may include OCT volume images of tissues, so that the model 132 is capable of identifying and/or discovering biomarkers associated with a health status category of the tissues (e.g., diseases, conditions, disease progressions, etc.) from a test dataset of OCT volume images of said tissues. In some instances, the health status category of a tissue may range from healthy to the various stages of a disease. For example, the health status categories associated with a retina can range from healthy to the various stages of AMD, including but not limited to early AMD, intermediate AMD, nascent GA, etc. In some instances, different biomarkers may be associated with the different health status categories of a disease.

For example, AMID is a leading cause of vision loss in patients 50 years or older. Initially, AMD manifests as a dry type of AMD before progressing to a wet type at a later stage. For the dry type, small deposits, called drusen, form beneath the basement membrane of the retinal pigment epithelium (RPE) and the inner collagenous layer of the Bruch's membrane (BM) of the retina, causing the retina to deteriorate in time. In its advanced stage, dry AMD can appear as geographic atrophy (GA), which is characterized by progressive and irreversible loss of choriocapillaries, RPE, and photoreceptors. Wet type AMD manifests with abnormal blood vessels originating in the choroid layer of the eye growing into the retina and leaking fluid from the blood into the retina. As such, in some embodiments, drusen may be considered biomarkers of one type of health status category of AMD (e.g., the dry type of AMD), while a missing RPE may be considered a biomarker of another type of health status category of AMD (e.g., the wet type of AIMD). It is to be noted that other health status categories (e.g., intermediate AMD, nascent GA, etc.) may be defined for AMD (e.g., or other types of retinal diseases) and that at least one or more differentiable biomarkers may be associated with these health status categories.

As noted above, morphological changes to, and/or the appearance of new, regions, boundaries, etc., in a retina or an eye may be considered as biomarkers of the retinal diseases such as AMD. Examples of such morphological changes may include distortions (e.g., shape, size, etc.), attenuations, abnormalities, missing or absent regions/boundaries, defects, lesions, and/or the like. For instance, as mentioned above, a missing RPE may be indicative of a retinal degenerative disease such as AMD. As another example, the appearance of regions, boundaries therebetween, etc., that are not present in a healthy eye or retina, such as deposits (e.g., drusen), leaks, etc., may also be considered as biomarkers of retinal diseases such as AMD. Other examples of features in a retina that may be considered as biomarkers include a reticular pseudodrusen (RPD), a retinal hyperreflective foci (e.g., a lesion with equal or greater reflectivity than the RPE), a hyporeflective wedge-shaped structure (e.g., appearing within the boundaries of the OPL), choroidal hypertransmission defects, and/or the like.

Output generator 136 may generate other forms of output. For example, in one or more embodiments, output generator may generate a report 150 to be displayed on display system 106 or to be sent over network 120 or another network to a remote device (e.g., cloud, mobile device, laptop, tablet, etc.). The report 150 may include, for example, without limitation, the OCT volume image 114, the saliency volume image, the map output for the OCT volume image, a list of any identified biomarkers, a treatment recommendation for the retina of the subject, an evaluation recommendation, a monitoring recommendation, some other type of recommendation or instruction, or a combination thereof. The monitoring recommendation may, for example, include a plan for monitoring the retina of the subject and a schedule for future OCT imaging appointments. The evaluation recommendation may include, for example, a recommendation to further review (e.g., manually review by a human reviewer) a subset of the plurality of OCT slice images that form the OCT volume image. The subset identified may include fewer than 5% of the plurality of OCT slice images. In some cases, the subset may include fewer than 50%, 45%, 40%, 35%, 30%, 25%, 20%, 15%, 10%, 5%, 2%, or some other percentage of the plurality of OCT slice images.

In one or more embodiments, the health status identification system 101 stores the OCT volume image 114 obtained from the OCT imaging system 110, saliency map 144, map output 146, an identification of the set of biomarkers 142, report 150, other data generated during the processing of the OCT volume image 114, or a combination thereof in data storage 104. In some embodiments, the portion of data storage 104 storing such information may be configured to comply with the security requirements of the Health Insurance Portability and Accountability Act (HIPAA) that mandate certain security procedures when handling patient data (e.g., such as OCT images of tissues of patients), i.e., the data storage 104 may be HIPAA-compliant. For instance, the information being stored may be encrypted and anonymized. For example, the OCT volume image 114 may be encrypted as well as processed to remove and/or obfuscate personally identifying information (PII) of the subjects from which the OCT volume image 114 was obtained. In some instances, the communications link between the OCT imaging system 110 and the health status identification system 101 that utilizes the network 120 may also be HIPAA-compliant. For example, the communication links may be a virtual private network (VPN) that is end-to-end encrypted and configured to anonymize PII data transmitted therein.

In one or more embodiments, the health identification system 101 includes a system interface 160 that enables human reviewers to interact with the images, maps, and/or other outputs generated by the health identification system 101. The system interface 160 may include, for example, but is not limited to, a web browser, an application interface, a web-based user interface, some other type of interface component, or a combination thereof.

Although the discussion herein is generally directed to the classification of OCT volume images (and OCT slice images) with respect to stages of AMD and the identification and discovery of biomarkers of AMD from OCT volume images (or OCT slice images) of retinas, the discussion may equally apply to medical images of other tissues of a subject obtained using any other medical imaging technology. That is, the OCT volume image 114 and the related discussion about the steps for classifying the OCT volume image 114 and for the identification and/or discovery of AMD biomarkers via the generation of saliency maps (e.g., heatmaps) of the retinal OCT slice images are intended as non-limiting illustrations, and same or substantially similar method steps may apply for the identification and/or discovery of other tissue diseases from 3D images (e.g., OCT or otherwise) of the tissues.

### II.B. Example Methodologies for Health Status Identification

FIG. 2 is a flowchart of a process for processing an OCT volume image of a retina of a subject to determine whether the OCT volume image evidences a selected health status category for the retina in accordance with one or more example embodiments. Process 200 in FIG. 2 may be implemented using health status identification system 101 in FIG. 1. For example, at least some of the steps of the process 200 may be performed by the processors of a computer or a server implemented as part of health status identification system 101. Process 200 may be implemented using model 132, saliency mapping algorithm 134, and/or output generator 136 in FIG. 1. Further, it is understood that additional steps may be performed before, during, or after the steps of process 200 discussed below. In addition, in some embodiments, one or more of the steps may also be omitted or performed in different orders.

Process 200 may optionally include the step 201 of training a deep learning model. The deep learning model may be one example of an implementation for model 132 in FIG. 1. The deep learning model may include, for example, without limitation, a neural network model. The deep learning model may be trained on a training dataset such as, for example, without limitation, training dataset 148 in FIG. 1. Examples of how the deep learning model may be trained are described in further detail below in Section II.D.

Step 202 of process 200 includes receiving an optical coherence tomography (OCT) volume image of a retina of a subject. The OCT volume image may be, for example, OCT volume image 114 in FIG. 1. The OCT volume image may be comprised of a plurality of OCT slice images.

Step 204 includes generating, via a deep learning model, a health indication output using the OCT volume image in which the health indication output indicates a level of association between the OCT volume image and a selected health status category for the retina. The health indication output may be, for example, health indication output 138 in FIG. 1. In one or more embodiments, the health indication output is a classification score. The classification score may be, for example, a probability that the OCT volume image, and thereby the retina captured in the OCT volume image, can be classified as being of the selected health status category. In other words, the classification score may be the probability that the OCT volume images evidences the selected health status category for the retina. In some embodiments, a threshold for the probability score (e.g., >0.5, >0.6, >0.7, >0.75, >0.8, etc.) is used to determine whether the OCT volume image evidences the selected health status category or not.

The selected health status category may be, for example, selected health status category 140 in FIG. 1. In one or more embodiments, the selected health status category represents a current health status for the retina (e.g., a current disease state). In one or more other embodiments, the selected health status category represents a future health status (e.g., a future disease state that is predicted to develop within a selected period of time). For example, the selected health status category may represent nascent GA that is either currently present or predicted to develop within a selected period of time (e.g., 3 months, 6 months, 1 year, 2 years, 3 years, or some other period of time).

The deep learning model may generate the health indication output in different ways. In one or more embodiments, the deep learning model generates an initial output for each OCT slice image in the OCT volume image to form a plurality of initial outputs. The initial output for an OCT slice image may be, for example, without limitation, a probability that the OCT slice image evidences the selected health status category for the retina. The deep learning model may use the plurality of initial outputs to generate the health indication output. For example, the deep learning model may average the plurality of initial outputs together to generate a health indication output that is a probability that the OCT volume image as a whole evidences the selected health status category for the retina. In other words, the health indication output may be a probability that the retina can be classified with the selected health status category. In other embodiments, the median of the plurality of initial outputs may be used as the health indication output. In still other embodiments, the plurality of initial outputs may be combined or integrated in some other manner to generate the health indication output.

Step 206 includes generating a map output (e.g., map output 146) for the deep learning model using a saliency mapping algorithm, wherein the map output indicates a level of contribution of a set of regions in the OCT volume image to the health indication output generated by the deep learning model. The level of contribution of a region in the OCT volume may be the degree of importance or impact that the region has on the health indication output generated by the deep learning model. This region may be defined as a single pixel or multiple pixels. The region may continuous or discontinuous. In one or more embodiments, the saliency mapping algorithm receives data from the deep learning model. This data may include, for example, features, weights, or gradients used by the deep learning model to generate the health indication output in step 204. The saliency map algorithm may be used to generate a saliency map (or heatmap) that indicates a degree of importance for the various portions of the OCT volume image with respect to the selected health status category (which is the class of interest).

For example, the saliency mapping algorithm may generate a saliency map for each OCT slice image of the OCT volume image. In one or more embodiments, the saliency mapping algorithm is implemented using Grad-CAM. The saliency map may be, for example, a heatmap that indicates the level of contribution (or degree of importance) of each pixel in the corresponding OCT slice image to the health indication output generated by the deep learning model with respect to the selected health status category. The saliency maps together for the plurality of OCT slice images in the OCT volume image may form a saliency volume map. The saliency maps may use color, annotations, text, highlighting, shading, patterns, or some other type of visual indicator to indicate degree of importance. In one example, a range of colors may be used to indicate a range of degrees of importance.

The saliency volume map may be used to generate the map output in various ways. In one or more embodiments, each saliency map for each OCT slice image may be filtered to generate a modified saliency map. For example, one or more filters (e.g., threshold, processing filters, numerical filters, color filters, shading filters, etc.) may be applied to the saliency maps to generate modified saliency maps that together form a modified saliency volume map. Each modified saliency map may visually signal the most important regions of the corresponding OCT slice image. In one or more embodiments, each modified saliency map is overlaid over its corresponding OCT slice image to generate the map output. For example, a modified saliency map may be overlaid over the corresponding OCT slice image such that the portion(s) of the OCT slice image determined to be most important (or relevant) to the model for the selected health status category is indicated. In one or more embodiments, the map output includes all of the overlaid OCT slice images. In one or more embodiments, the map output may provide a visual indication on each overlaid OCT slice image of the regions having the most important or impactful contribution to the generation of the health indication output.

In other embodiments, the modified saliency maps are processed in another manner to generate a map output that indicates which regions of the OCT slice images are most impactful to the model for the selected health status category. For example, information from the modified saliency maps may be used to annotate and/or otherwise graphically modify the corresponding OCT slice images to form the map output.

Process 200 may optionally include step 208. Step 208 includes identifying a set of biomarkers (e.g., set of biomarkers 142 in FIG. 1) in the OCT volume image for the selected health status category using the map output. Step 208 may be performed in different ways. In one or more embodiments, a potential biomarker region may be identified in association with a selected region of an OCT slice image identified by the map output as being important or impactful to the selected health status category. The potential biomarker region may be identified as this selected region of the OCT slice image or may be defined based on this selected region of OCT slice image. In one or more embodiments, a bounding box is created around the selected region of the OCT slice image to define the potential biomarker region.

A scoring metric may be generated for the potential biomarker region. The scoring metric may include, for example, a size of the potential biomarker region, a confidence score for the potential biomarker region, some other metric, or a combination thereof. The potential biomarker region (e.g., bounding box) may be identified as a biomarker for the selected health status category when the scoring metric meets a selected threshold. For example, if the scoring metric includes a confidence score and dimensions, then the selected threshold may include a confidence score threshold (e.g., score minimum) and minimum dimensions. In some embodiments, a particular biomarker may be found on or span multiple OCT slice images. In one or more embodiments, the bounding boxes that meet the threshold and that are classified as biomarker regions may be identified on the corresponding OCT slice images to form biomarker maps.

One or more of the biomarkers that are identified may be known biomarkers that have been previously seen by human reviewers. In some embodiments, one or more of the biomarkers may be new, not previously known biomarkers. In other words, the identification of the set of biomarkers in step 208 may include the discovery of one or more new biomarkers associated with the selected health status category. The discovery of one or more new biomarkers may be more prone to occur, for example, when the selected health status category represents a future health status that is predicted to develop (e.g., a future progression of AMD from early AMD or intermediate AMD to nascent GA; from nascent GA to GA; from intermediate AMD to GA, etc.).

Process 200 may optionally include step 210. Step 210 includes generating a report. The report may include, for example, without limitation, the OCT volume image, the saliency volume image, the map output for the OCT volume image, a list of any identified biomarkers, a treatment recommendation for the retina of the subject, an evaluation recommendation, a monitoring recommendation, some other type of recommendation or instruction, or a combination thereof. The monitoring recommendation may, for example, include a plan for monitoring the retina of the subject and a schedule for future OCT imaging appointments. The evaluation recommendation may include, for example, a recommendation to further review (e.g., manually review by a human reviewer) a subset of the plurality of OCT slice images that form the OCT volume image. The subset identified may include fewer than 5% of the plurality of OCT slice images. In some cases, the subset may include fewer than 50%, 45%, 40%, 35%, 30%, 25%, 20%, 15%, 10%, 5%, 2%, or some other percentage of the plurality of OCT slice images.

In some embodiments, the health identification system 101 in FIG. 1 may prompt (e.g., via an evaluation recommendation in report 150 in FIG. 1) user review of a particular subset of the OCT slice images within the OCT volume image to identify one or more features (or biomarkers) in the same or substantially similar locations as the bounding boxes identified on biomarker maps. For example, in some cases, the health identification system 101 may include a system interface 160 that allows reviewers (e.g., healthcare professionals, trained reviewers, etc.) to access, review and annotate the OCT slice images of the OCT volume image so as to identify and/or discover biomarkers. That is, for example, the system interface 160 may facilitate the annotation, by the reviewers, of the OCT slice images with biomarkers. In some instances, instead of or in addition to allowing reviewers to identify biomarkers on the OCT slice images shown in the biomarker maps, the system interface 160 may be configured to allow reviewers to correct or adjust the bounding boxes (e.g., adjust the size, shape, or continuity of the bounding boxes) on the biomarker maps. In some cases, the reviewers can annotate the bounding boxes to indicate the adjustments to be made. In some cases, the annotated and/or adjusted biomarker maps created by the reviewers may be fed back to the deep learning model (e.g., as part of the training dataset 148) for additional training of the deep learning model.

FIG. 3 is a flowchart of a process for identifying biomarkers in an OCT volume image of a retina of a subject in accordance with one or more example embodiments. Process 300 in FIG. 3 may be implemented using health status identification system 101 in FIG. 1. For example, at least some of the steps of the process 300 may be performed by the processors of a computer or a server implemented as part of health status identification system 101. Process 300 may be implemented using model 132, saliency mapping algorithm 134, and/or output generator 136 in FIG. 1. Further, it is understood that additional steps may be performed before, during, or after the steps of process 200 discussed below. In addition, in some embodiments, one or more of the steps may also be omitted or performed in different orders.

Process 300 may optionally include the step 302 of training a deep learning model. The deep learning model may be one example of an implementation for model 132 in FIG. 1. The deep learning model may include, for example, without limitation, a neural network model. The deep learning model may be trained on a training dataset such as, for example, without limitation, training dataset 148 in FIG. 1.

Step 304 of process 300 includes receiving an optical coherence tomography (OCT) volume image of a retina of a subject. The OCT volume image may be, for example, OCT volume image 114 in FIG. 1. The OCT volume image may be comprised of a plurality of OCT slice images.

Step 306 of process 300 includes generating, via a deep learning model, a health indication output using the OCT volume image in which the health indication output indicates a level of association between the OCT volume image and a selected health status category for the retina. For example, the health indication output may be a probability that indicates how likely the classification of the retina in the OCT volume image is the selected health status category. In other words, the health indication output may be probability that indicates how likely it is that the OCT volume image evidences the selected health status category for the retina.

Step 308 of process 300 includes generating a saliency volume map for the OCT volume image using a saliency mapping algorithm, wherein the saliency volume map indicates a level of contribution of a set of regions in the OCT volume image to the health indication output generated by the deep learning model. Step 308 may be performed in a manner similar to the generation of the saliency volume map described with respect to step 206 in FIG. 2. The saliency mapping algorithm may include, for example, a Grad-CAM algorithm. The level of contribution may be determined based on the features, gradients, or weights used in the deep learning model (e.g., the features, gradients, or weights used in the last activation layer of the deep learning model).

Step 310 of process 300 includes detecting a set of biomarkers for a selected health status category using the saliency volume map. Step 310 may be implemented in a manner similar to the identification of biomarkers described above with respect to step 208 in FIG. 2. For example, step 310 may include filtering the saliency volume map to generate a modified saliency volume map. The modified saliency volume map identifies a set of regions that are associated with the selected health status category. Step 310 may further include identifying a potential biomarker region in association with a region of the set of regions. A scoring metric may be generated for the potential biomarker region. The potential biomarker region may be identified as including at least one biomarker when the scoring metric meets a selected threshold.

II.C. Example Methodologies for Detecting Nascent Geographic Atrophy (nGA)

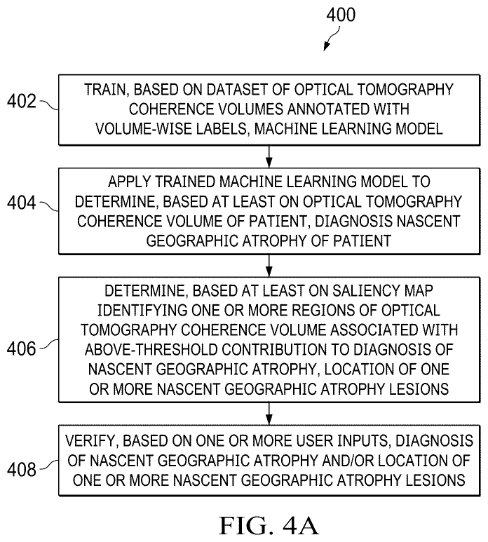

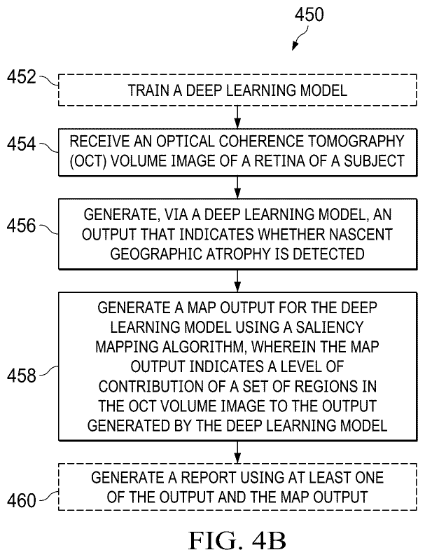

FIG. 4A is a flowchart of a process 400 for artificial intelligence assisted nascent geographic atrophy (nGA) detection in accordance with one or more example embodiments. The detection of nGA described with respect to FIG. 4A may include detection of one or more nGA lesions, localizing one or more nGA lesions, or a combination thereof. Such detection may be considered a diagnosis of nGA. The process 400 may be implemented using health status identification system 101 in FIG. 1. For example, at least some of the steps of the process 400 may be performed by the processors of a computer or a server implemented as part of health status identification system 101. Process 400 may be implemented using model 132, saliency mapping algorithm 134, and/or output generator 136 in FIG. 1. Further, it is understood that additional steps may be performed before, during, or after the steps of process 400 discussed below. In addition, in some embodiments, one or more of the steps may also be omitted or performed in different orders.

Step 402 of process 400 includes training, based on a dataset of OCT volumes, a machine learning model. For example, one implementation of step 402 may include using training data 148 in FIG. 1 and OCT volume image 114 in FIG. 1 to train a deep learning model. The deep learning model may be one example of an implementation for model 132 in FIG. 1.

Step 404 of process 400 includes applying the machine learning model to determine, based at least on OCT volumes of a patient, a diagnosis of nascent geographic atrophy of a patient. For example, one example of an implementation for step 404 may include using image processor 130 in FIG. 1 processing OCT imaging data 112 in FIG. 1 and health status identification system 101 to diagnose nGA. The diagnosis of nGA based on the OCT volumes may be one example of an implementation of step 204 in FIG. 2. The diagnosis of nGA may be based on a detection of one or more nGA lesions (e.g., detecting an onset of nGA based on detecting the presence of one or more nGA lesions).

Step 406 of process 400 includes determining, based on at least a saliency map identifying one or more regions of the OCT volume associated with an above-threshold contribution to diagnosis of nGA, a location of one or more nGA lesions. For example, one implementation of step 406 may include health status identification system 101 generating an output of saliency map 134 in FIG. 1 to identify a location of one or more nGA lesions.

Step 408 of process 400 includes verifying, based one or more inputs, a diagnosis of nGA and/or the locations of one more nGA lesions. An example implementation of step 408 may include health status identification system 101 verifying, based on one or more user inputs, the diagnosis of nascent geographic atrophy and/or the location of the one or more nascent geographic atrophy lesions.

FIG. 4B is a flowchart of a process for processing an OCT volume image of a retina of a subject to determine whether the OCT volume image evidences nascent geographic atrophy (nGA) in accordance with one or more example embodiments. Process 450 in FIG. 4B may be implemented using health status identification system 101 in FIG. 1. For example, at least some of the steps of the process 450 may be performed by the processors of a computer or a server implemented as part of health status identification system 101. Process 450 may be implemented using model 132, saliency mapping algorithm 134, and/or output generator 136 in FIG. 1. Further, it is understood that additional steps may be performed before, during, or after the steps of process 450 discussed below. In addition, in some embodiments, one or more of the steps may also be omitted or performed in different orders.

Process 450 may optionally include the step 452 of training a deep learning model. The deep learning model may be one example of an implementation for model 132 in FIG. 1. The deep learning model may include, for example, without limitation, a neural network model. The deep learning model may be trained on a training dataset such as, for example, without limitation, training dataset 148 in FIG. 1. Examples of how the deep learning model may be trained are described in further detail below in Section II.D.

Step 454 of process 450 includes receiving an optical coherence tomography (OCT) volume image of a retina of a subject. The OCT volume image may be, for example, OCT volume image 114 in FIG. 1. The OCT volume image may be comprised of a plurality of OCT slice images.

Step 456 includes generating, via a deep learning model, an output that indicates whether nascent geographic atrophy (nGA) is detected. This output may be, for example, one example of an implementation of health indication output 138 in FIG. 1. In one or more embodiments, the output is a classification score for nGA. The classification score may be, for example, a probability that the OCT volume image, and thereby the retina captured in the OCT volume image, can be classified as evidencing nGA (e.g., evidencing an onset of nGA or another substage of nGA). In other words, the classification score may be the probability that the OCT volume images evidences nGA for the retina. In some embodiments, a threshold for the probability score (e.g., >0.5, >0.6, >0.7, >0.75, >0.8, etc.) is used to determine whether the OCT volume image evidences nGA. Step 456 may be implemented in a manner similar to the implementation of step 204 described with respect to FIG. 2.

Step 458 includes generating a map output (e.g., map output 146) for the deep learning model using a saliency mapping algorithm, wherein the map output indicates a level of contribution of a set of regions in the OCT volume image to the output generated by the deep learning model. The level of contribution of a region in the OCT volume may be the degree of importance or impact that the region has on the output generated by the deep learning model.

This region may be defined as a single pixel or multiple pixels. The region may continuous or discontinuous. In one or more embodiments, the saliency mapping algorithm receives data from the deep learning model. This data may include, for example, features, weights, or gradients used by the deep learning model to generate the output in step 456. The saliency map algorithm may be used to generate a saliency map (or heatmap) that indicates a degree of importance for the various portions of the OCT volume image with respect to the selected health status category (which is the class of interest).

For example, the saliency mapping algorithm may generate a saliency map for each OCT slice image of the OCT volume image. In one or more embodiments, the saliency mapping algorithm is implemented using Grad-CAM. The saliency map may be, for example, a heatmap that indicates the level of contribution (or degree of importance) of each pixel in the corresponding OCT slice image to the health indication output generated by the deep learning model with respect to the selected health status category. The saliency maps together for the plurality of OCT slice images in the OCT volume image may form a saliency volume map. The saliency maps may use color, annotations, text, highlighting, shading, patterns, or some other type of visual indicator to indicate degree of importance. In one example, a range of colors may be used to indicate a range of degrees of importance.

The saliency volume map may be used to generate the map output in various ways. In one or more embodiments, each saliency map for each OCT slice image may be filtered to generate a modified saliency map. For example, one or more filters (e.g., threshold, processing filters, numerical filters, color filters, shading filters, etc.) may be applied to the saliency maps to generate modified saliency maps that together form a modified saliency volume map. Each modified saliency map may visually signal the most important regions of the corresponding OCT slice image. In one or more embodiments, each modified saliency map is overlaid over its corresponding OCT slice image to generate the map output. For example, a modified saliency map may be overlaid over the corresponding OCT slice image such that the portion(s) of the OCT slice image determined to be most important (or relevant) to the model for the selected health status category is indicated. In one or more embodiments, the map output includes all of the overlaid OCT slice images. In one or more embodiments, the map output may provide a visual indication on each overlaid OCT slice image of the regions having the most important or impactful contribution to the generation of the output.

In other embodiments, the modified saliency maps are processed in another manner to generate a map output that indicates which regions of the OCT slice images are most impactful to the model for the selected health status category. For example, information from the modified saliency maps may be used to annotate and/or otherwise graphically modify the corresponding OCT slice images to form the map output.

The one or more regions identified by the map output may indicate, for example, directly correspond with one or more nGA lesions. For example, a region identified in the map output may be considered the location of one or more nGA lesions.

In one or more embodiments, the map output may be annotated with other information. For example, the map output may include a bounding box that is created around a selected region of an OCT slice image that is identified as an nGA lesion or evidencing one or more nGA lesions. In some cases, the bounding box may be annotated with a scoring metric (e.g., a confidence score, dimensions, etc.). In one or more embodiments, bounding boxes meeting threshold dimensions, meeting a threshold confidence score, or both are classified as evidencing nGA.

Process 450 may optionally include step 460. Step 460 includes generating a report. The report may include, for example, without limitation, the OCT volume image, the saliency volume image, the map output for the OCT volume image, a list of any identified biomarkers, a treatment recommendation for the retina of the subject, an evaluation recommendation, a monitoring recommendation, some other type of recommendation or instruction, or a combination thereof. The monitoring recommendation may, for example, include a plan for monitoring the retina of the subject and a schedule for future OCT imaging appointments. The evaluation recommendation may include, for example, a recommendation to further review (e.g., manually review by a human reviewer) a subset of the plurality of OCT slice images that form the OCT volume image. The subset identified may include fewer than 5% of the plurality of OCT slice images. In some cases, the subset may include fewer than 50%, 45%, 40%, 35%, 30%, 25%, 45%, 15%, 10%, 5%, 2%, or some other percentage of the plurality of OCT slice images.

In some embodiments, the health identification system 101 in FIG. 1 may prompt (e.g., via an evaluation recommendation in report 150 in FIG. 1) user review of a particular subset of the OCT slice images within the OCT volume image to identify one or more features (or biomarkers) in the same or substantially similar locations as the bounding boxes identified on biomarker maps. For example, in some cases, the health identification system 101 may include a system interface 160 that allows reviewers (e.g., healthcare professionals, trained reviewers, etc.) to access, review and annotate the OCT slice images of the OCT volume image so as to identify and/or discover biomarkers. That is, for example, the system interface 160 may facilitate the annotation, by the reviewers, of the OCT slice images with biomarkers. In some instances, instead of or in addition to allowing reviewers to identify biomarkers on the OCT slice images shown in the biomarker maps, the system interface 160 may be configured to allow reviewers to correct or adjust the bounding boxes (e.g., adjust the size, shape, or continuity of the bounding boxes) on the biomarker maps. In some cases, the reviewers can annotate the bounding boxes to indicate the adjustments to be made. In some cases, the annotated and/or adjusted biomarker maps created by the reviewers may be fed back to the deep learning model (e.g., as part of the training dataset 148) for additional training of the deep learning model.

### II.D. Exemplary Training of Deep Learning Model

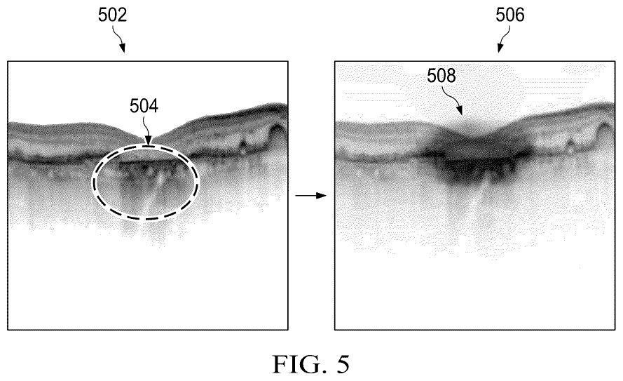

The deep learning models described above in FIG. 1 (e.g., model 132), FIG. 2, FIG. 3, FIG. 4, and FIG. 5 may be trained in different ways. In one or more embodiments, the deep learning model is trained with a training dataset (e.g., training dataset 148 in FIG. 1) that includes one or more training OCT volume images. Each of these training OCT volume images may be of a different retina that has been identified as displaying a disease or a condition corresponding to the selected health status category (e.g., nascent GA, etc.). The retinas may have been displaying the disease or condition for a length of time at least substantially equal to the duration after the training OCT volume image is taken or generated.

In one or more embodiments, the deep learning model (e.g., model 132 in FIG. 1, the deep learning model described in FIGS. 2-3) may be trained to classify the health status of a retina based on a training dataset (e.g., training dataset 148 in FIG. 1) of OCT volume images of retinas of patients suffering from one or more health status categories so that the deep learning model may learn what features of the retina, and locations thereof, in the OCT volume images are signals for the one or more health status categories. When provided with a test dataset of OCT volume images, the trained deep learning model may then be able to efficiently and accurately identify whether the OCT volume images evidence a selected health status category.

A training dataset of OCT volume images may include OCT images of retinas of patients that are known to be suffering from a given stage of AMD (i.e., the health status category of the retinas may be the said stage of AMD). In such cases, the deep learning model may be trained with the training dataset to learn what features in the OCT volume images correspond to, are associated with, or signal that stage of AMD. For example, the patients may be sufferers of late-stage AMD, and the deep learning model may identify, or discover, from the training dataset of OCT volume images of the patients' retinas that the anatomical features in an OCT volume image representing a severely deformed RPE may be evidence of late-stage AMD. In such cases, when provided with an OCT volume image of a retina of a patient showing a severely deformed RPE, the trained deep learning model may identify the OCT volume image as one that belongs to a late-stage AMD patient.

In some embodiments, the deep learning model may be capable of classifying health status even based on unknown biomarkers of retinal diseases. For example, the deep learning model may be provided with a training dataset of OCT volume images of retinas of patients that are suffering from some retinal disease (e.g., nascent GA) all the biomarkers of which may not be known. That is, the biomarkers for that selected health status category (e.g., nascent GA) of the retinal disease may not be known. In such cases, the deep learning model may process the dataset of OCT volume images and learn that a feature or a pattern in the OCT volume images, e.g., lesions, is evidence of the selected health status category.

### III. Example OCT Images and Example Map Outputs

FIG. 5 illustrates an annotated OCT slice image and a corresponding heatmap for the annotated OCT slice image in accordance with one or more example embodiments. The OCT slice image 502 may be one example of an implementation for an OCT slice image in OCT volume image 114 in FIG. 1. The OCT slice image 502 may also be one example of an implementation from an OCT slice image in training dataset 148 in FIG. 1. OCT slice image 502 includes annotated region 504 that has been marked by a human grader as being a biomarker for nascent GA.

Heatmap 506 is one example of an implementation for at least a portion of map output 146 in FIG. 1. Heatmap 506 may be the result of overlaying a saliency map generated using a saliency mapping algorithm such as saliency mapping algorithm 134 in FIG. 1 (e.g., generated using Grad-CAM) over OCT slice image 502. The saliency map was generated for a trained deep learning model that processed OCT slice image 502. Heatmap 506 indicates that region 508 was most impactful to the model for nascent GA and shows that the deep learning model, which may be, for example, model 132 in FIG. 1, accurately used the correct region of the OCT slice image 502 for its classification with respect to nascent GA.

Heatmap 506 may be used to identify and localize the biomarker shown within region 508 for nascent GA. For example, an output may be generated that identifies an anatomic biomarker located within region 508. The biomarker may be, for example, a lesion in the retina, missing retinal pigment epithelium (RPE), a detached layer of the retina, or some other type of biomarker. In some cases, more than one biomarker may be present within region 508. In some instances, filtering may be performed to identify certain pixels within region 508 of heatmap 506 or within region 504 of the OCT slice image 502 that are a biomarker. In some instances, the size of region 508 may be used to determine whether region 508 contains one or more biomarkers. In one or more embodiments, the size of region 508 is greater than about 20 pixels.

Such identification and localization of biomarkers may allow a healthcare practitioner to diagnose, monitor, treat, etc., the patient whose retina is depicted in the OCT slice image 502. For example, an ophthalmologist reviewing the heatmap 506 or information generated based on the heatmap 506 may be able to recommend a treatment option or monitoring option prior to the onset of GA.

FIG. 6 is an illustration of different maps in accordance with one or more example embodiments. Saliency map 602 is one example of an implementation for a saliency map that makes up saliency volume map 144 in FIG. 1. Modified saliency map 604 is one example of an implementation for a saliency map that has been modified after filtering (e.g., applying a threshold filter). Heatmap 606 is one example of an implementation for a component of map output 146 in FIG. 1. Heatmap 606 includes a modified overlay of saliency map 602 over an OCT slice image.

Biomarker map 608 is an example of an implementation for an output that may be generated by output generator 136 in FIG. 1 using heatmap 606. In biomarker map 608, a first bounding box identifies a potential biomarker region that does not have a sufficiently high confidence score (e.g., >0.6) to be considered a biomarker region that includes at least one biomarker. Further, biomarker map 608 includes a second bounding box that identifies a potential biomarker region that has a sufficiently high confidence score to be considered a biomarker region that includes at least one biomarker.

The images and map outputs (e.g., heatmaps, saliency maps) depicted in FIGS. 5-6 below are shown with one example grayscale. In other embodiments, other grayscales may be used. For example, an OCT image such as depicted in FIG. 5 may have a grayscale that is inverted or partially inverted with respect to the grayscale depicted in FIG. 5. As one non-limiting example, background that is shown in white in FIG. 5 may be black in other example embodiments. In some embodiments, a range of colors may be used to generate the map outputs. For example, the map outputs shown in grayscale in FIG. 6 may be colored in other embodiments. In other embodiments, the biomarker maps shown in FIG. 6 may be annotated with color, may have potential biomarker regions identified via color, or both.

### IV. Example System for Nascent Geographic Atrophy Detection

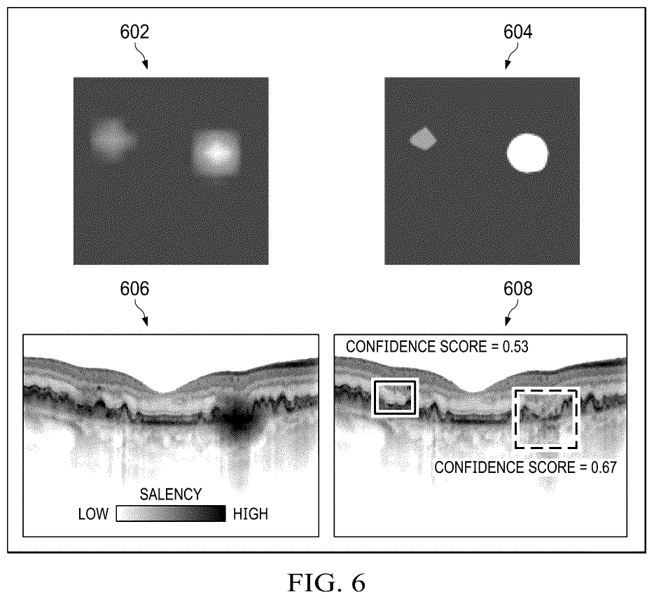

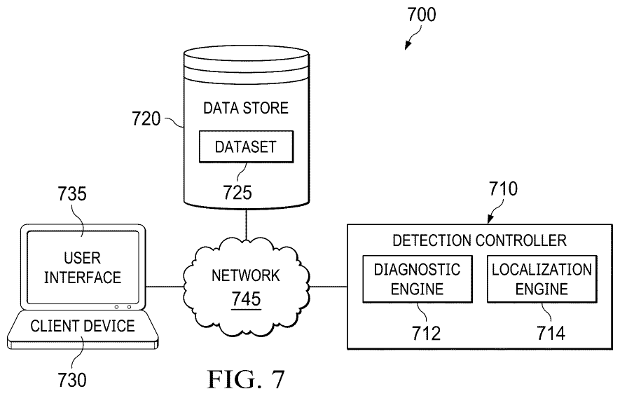

FIGS. 7-13 describe a system for nascent geographic atrophy (nGA) detection and various workflows using that system. This nascent geographic atrophy detection system 700 may be one example of an implementation for health status indication system 101 in FIG. 1. Training is described with respect to one or more different types of example training datasets.

FIG. 7 depicts a system diagram illustrating an example of a nascent geographic atrophy detection system 700, in accordance with some example embodiments. The nascent geographic atrophy detection system 700 may include a detection controller 710 including a diagnostic engine 712 and a localization engine 714, a data store 720, and a client device 730. The detection controller 710, the data store 720, and the client device 730 may be communicatively couple via a network 740. The detection controller may be one example of a full or partial implementation of image processor 130 in FIG. 1.

The client device 730 may be a processor-based device including, for example, a mobile device, a wearable apparatus, a personal computer, a workstation, an Internet-of-Things (IoT) appliance, and/or the like. The data store 720 may be a database including, for example, a non-relational database, a relational database, an in-memory database, a graph database, a key-value store, a document store, and/or the like. Data store 720 may be one example implementation of data storage 104 in FIG. 1. The network 745 may be a wired network and/or wireless network including, for example, a public land mobile network (PLMN), a local area network (LAN), a virtual local area network (VLAN), a wide area network (WAN), the Internet, and/or the like. Network 745 may be one example of an implementation of network 120 in FIG. 1.

In some example embodiments, the diagnostic engine 712 may be implemented using a deep learning model such as, for instance, an artificial neural network (ANN) based classifier. Diagnostic engine 712 may be one example of an implementation of health status model 132 in FIG. 1. In some cases, the diagnostic engine 712 may be implemented as a residual neural network (ResNet) based classifier. The diagnostic engine 712 may be configured to perform nascent geographic atrophy (nGA) diagnoses that includes determining, based at least on one or more optical coherence tomography (OCT) volumes of a patient, whether the patient exhibits nascent geographic atrophy. Lesion localization to identify the location of one or more nascent geographic atrophy (nGA) lesions may be performed based on a visual explanation of the deep learning model applied by the diagnostic engine 712. For example, the localization engine 714 may be configured to perform nascent geographic atrophy (nGA) lesion localization that includes determining, based at least on a saliency map identifying regions of the optical coherence tomography (OCT) volumes associated with an above-threshold contribution to a diagnosis of a nascent geographic atrophy, a location of one or more lesions associated with nascent geographic atrophy (nGA). The saliency map may be generated by applying, for example, a gradient weighted class activation mapping (GradCAM), which outputs a heatmap of how much each region within an image, such as an optical coherence tomography (OCT) volume, contributes to the class label ultimately assigned to the image.

In some example embodiments, the deep learning model implementing the diagnostic engine 712 may be trained on a dataset 725 stored, for example, in the data store 720. Dataset 725 may be one example implementation of training dataset 148 in FIG. 1. In one example, the dataset 725 includes, but is not limited to, a total of 1,884 optical coherence tomography volumes from 280 eyes of 740 subjects with intermediate age-related macular degeneration (iAMD). Overall, 1,766 optical coherence tomography volumes were labeled as without nascent geographic atrophy (i.e., no nGA detected) and 118 volumes were labeled as with nascent geographic atrophy (i.e., nGA detected). As used herein, a diagnosis of nascent geographic atrophy may also include nascent geographic atrophy that enlarges to the size that would meet the criteria for a diagnosis of complete retinal pigment epithelial and outer retinal atrophy (cRORA).

The optical coherence tomography volumes may be further labeled with the location of nascent geographic atrophy lesions, for example, with bounding boxes horizontally covering the subsidence, vertically starting at inner limiting layer (ILL) layer and stopping at retinal pigment epithelium (RPE) layer. The bounding boxes may be used in evaluating the weakly supervised lesion localization (e.g., performed by the localization engine 714) and not in model training. Since the dataset 725 for training the deep leaning model includes class labels of 3D optical coherence tomography volume, the training of the deep learning model to perform nascent geographic atrophy (nGA) diagnosis and lesion localization may be considered weakly supervised.

IV.A. Example Model for Classifying OCT Volumes with Respect to nGA

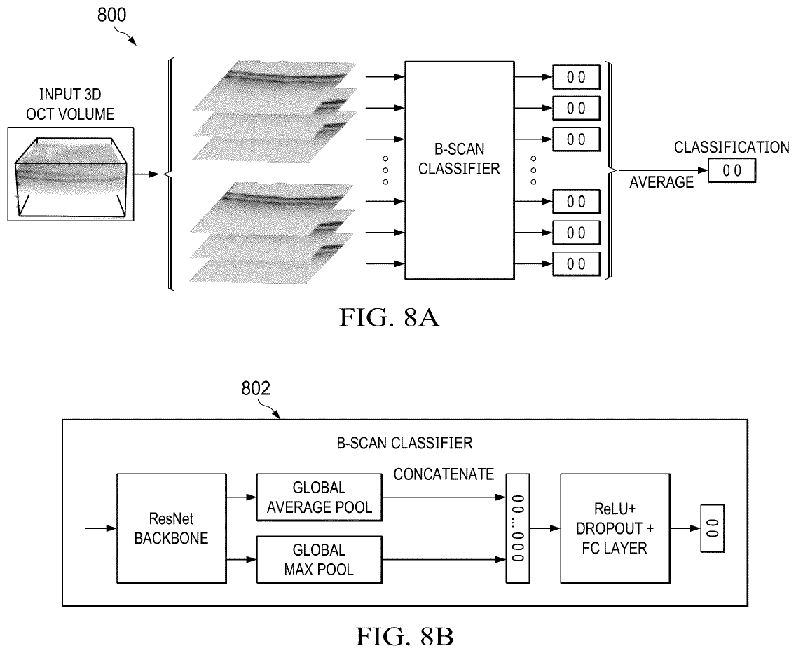

FIGS. 8A-8B depict an example of the deep learning architecture implementing the diagnostic engine 712 and the localization engine 714 of the detection controller 710 of the nascent geographic atrophy detection system 700 shown in FIG. 7. The components shown in FIGS. 8A-8B may be examples of components used to implement health status identification system 101 in FIG. 1.

FIG. 8A is an illustration of one example of a model 800 for processing a 3D OCT volume in accordance with one or more example embodiments. The model 800, which may include a deep neural network based OCT B-scan classifier, is used to generate a classification score that indicates whether nGA is detected in OCT volume.

FIG. 8B illustrates one example of an implementation for a classifier 802 that may be used to implement the classifier in model 800 in FIG. 8A in accordance with one or more example embodiments. Classifier 802 may be used to classify OCT B-scans. Classifier 802 may be implemented using a residual neural network (ResNet) backbone whose output is coupled with a rectifier linear unit (ReLU) and a fully connected (FC) layer. A late-fusion method with the residual neural network (ResNet) backbone may be applied to the 3D OCT volumes.

As an example, in FIG. 8A, B-scans are fed into a B-scan classifier of the model 800 and the outputs are vectors of classification logits for each B-scan. The B-scan logits are averaged to generate a classification score for each OCT volume. Thinking of the B-scans as instances and the OCT volumes as bags, this framework can be categorized as an example of multi-instance learning in which the model 800 is trained on weakly labeled data, using labels on bags (OCT volumes). During the training process, given an OCT volume annotated as nascent geographic atrophy, the model 800 may be forced to identify as many as B-scans with nascent geographic atrophy lesion to improve the final prediction of nGA, thus the trained model allows prediction of nGA labels on OCT volumes as well as on individual B-scans.

The details of an example B-scan classifier 802 are shown in FIG. 8B. For example, an individual B-scan of size 512×496 from the volume is passed through the residual neural network (e.g., ResNet-18) backbone, which outputs activation maps (e.g., 512×16×16 activation map). A max-pooling layer and an average pooling layer can be applied to the output of the residual neural network before their respective outputs are concatenated to generate a feature vector (e.g., a 1024 long feature vector). A fully connected layer may then be applied to the feature vector generate the classification logit vector corresponding to a categorical distribution for the B-scan.

### IV.B. Example Training of Model

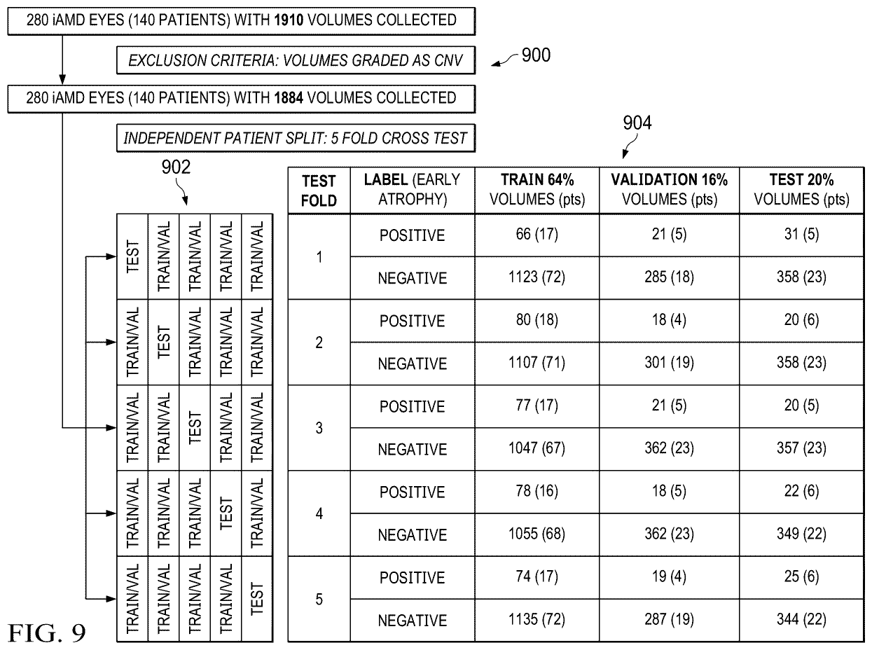

FIG. 9 depicts an exemplary data flow diagram with data split statistics in accordance with one or more example embodiments. This data flow diagram tracks the training of a model (e.g., model 800 in FIGS. 8A-8B) based on one example of data collected for various subjects as part of an experiment or study. Training data 900, which may be one example of the dataset 725 in FIG. 7, is generated from 1,910 OCT volumes from 280 eyes of 140 intermediate age-related macular degeneration (iAMD) participants, with 1 volume per eye per semi-annual visit for up to 3 years. Volumes graded as neovascular age-related macular degeneration were excluded. In the remaining 1,884 volumes, 118 volumes from 40 eyes of 28 participants were graded as being positive for nGA. 5-fold cross-validation 902 was performed on the training data 900, with 5 models being trained in 5 different splits, in a “cross-validation” fashion. In each split, the training data 900 was split into a training set, a validation set, and a test set, by patient. Early stopping was applied for monitoring F1 score on the validation set. Model performance evaluation was applied on the test set. Table 904 provides split statistics, number of volumes, and participants for the 5-fold cross validation 902. It should be appreciated that the number of eyes is twice the number of participants.

Where the training data 900 covers a small number of participants, the performance of the deep learning model may be tested on the entire dataset, in 5 test sets from 5 different folds of splits. For each fold, the test set of optical coherence tomography volumes were obtained from roughly 20% of the participants stratified on whether the patient developed nGA. The OCT volumes from the remaining 80% participants were further split into training (64%) and validation sets (16%), with volumes from one patient only existing in one of the sets. The corresponding test set was not used in the training and validation process, even though the term cross-validation was used to describe the data splits.

In some example embodiments, at least some pre-processing may be performed on B-scans for standardization. For example, the B-scans may be resized (e.g., to 512×496 pixels) before being rescaled to an intensity range of [0, 1]. Some data augmentation, such as rotation of small angles, horizontal flips, vertical flips, addition of Gaussian noises, and Gaussian blur, may be randomly applied to improve the model's invariance to those transformations.

Referring again to FIG. 8B, the residual neural network (ResNet) backbone of the classifier 802 may be pre-trained on an ImageNet dataset. In one or more embodiments, during model training, an Adam optimizer may be used to minimize focal loss while an L2 weight decay regularization may be applied to improve the model's ability to generalize across the training data 900. In some cases, hyper-parameter tuning may be performed using the training data 900 and validation set to find the optimal value of learning rate and weight decay. The model 800, trained with the optimal hyper-parameter, may be tested on the test set. Various metrics may be evaluated to indicate model performance. Such metrics include, for example, but are not limited to, area under the curve (AUC), area under the precision-recall curve (AUPRC), recall, precision, and F1-score. Additionally, a confusion matrix may be computed.

### IV.C. Example Generation of Map Outputs

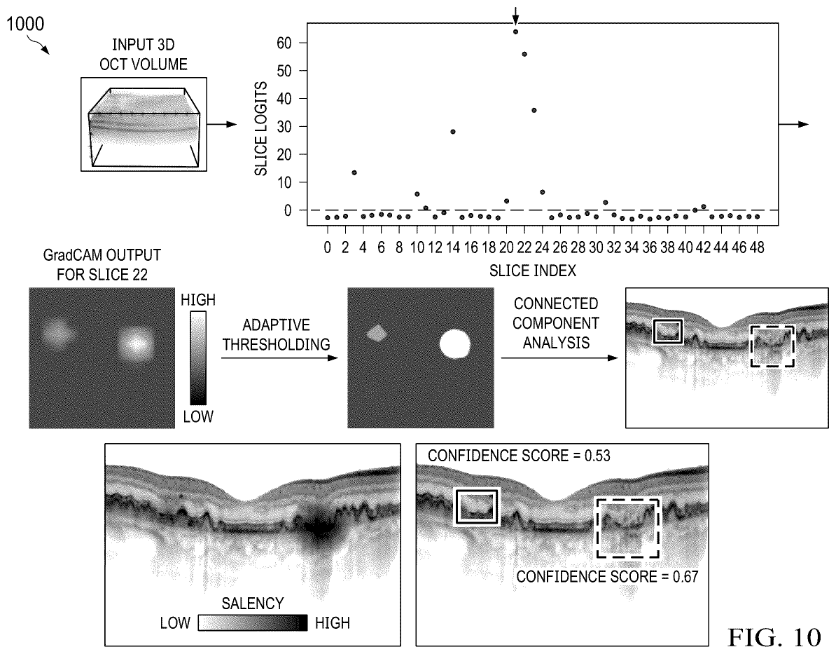

FIG. 10 is an illustration of an output workflow for outputs generated from an OCT volume in accordance with one or more example embodiments. In workflow 1000, the output of the gradient weighted class activation mapping may be overlaid on the input Oct images for easy visualization of the saliency as well as the original grayscale OCT image. Areas that are visually emphasized (e.g., via specific coloring or highlighting) may indicate the location(s) of nGA lesions. Saliency maps may be used to reason the model's (e.g., model 800) decision, check the model's generalizability, as well as examine and leverage the model's ability in nascent geographic atrophy lesion detection.

As shown in workflow 1000, B-scan logits for individual OCT B-scans of the OCT volume input into the model (e.g., model 800) are generated. These logits are used to classify the OCT volume as evidencing nGA or not. The GradCAM output for the model is shown for an individual slice (e.g., slice 22). Adaptive thresholding is applied to the corresponding GradCAM outputs of the gradient weighted class activation mapping (in the viridis colormap) before a bounding box is generated via connected component analysis. A confidence score of the bounding box may be estimated based on average saliency and the corresponding B-scan logit. A map output may be generated with the B-scan being overlaid with the GradCAM output, the B-scan being overlaid with bounding boxes and their associated confidence scores, or both. Bounding boxes having a confidence score below a threshold (e.g., <0.6) may be removed from subsequent processing.

The GradCAM output and the map output help visually localize nGA lesions. In one or more embodiments, each bounding box may be considered as potentially identifying an nGA lesion. For example, a bounding box with a confidence score above the threshold may be considered the location of one or more nGA lesions. In one or more embodiments, the confidence score for each bounding box may be estimated from the individual classification logit of the B-scan classifier as S(l/n h/Σh), wherein S denotes the sigmoid function, l denotes the individual B-scan classification logit, n denotes the quantity of B-scans in a volume, h denotes the mean saliency in the detected region, and Σh denotes the total mean saliency of all detected regions within the B-scan.

A higher confidence score may imply a higher probability that the detected region within a bounding box covers nascent geographic atrophy lesions. Accordingly, bounding boxes with below-threshold confidence scores (e.g., <0.6) may be removed by thresholding such that B-scans with one or more remaining bounding boxes (after the thresholding) may be identified as B-scans exhibiting nascent geographic atrophy (nGA).

In some example embodiments, the aforementioned confidence score threshold may be determined based on the B-scans with nascent geographic atrophy present in the validation set. For example, on the validation set, the number of classified nascent geographic atrophy B-scans and the recall of diagnosing nascent geographic atrophy B-scans with respect to different threshold values may be plotted, respectively. A lower threshold may cause the model to generate fewer false negatives (e.g., true nascent geographic atrophy B-scans that are misclassified as non-nascent geographic atrophy) and a greater number of false positives (e.g., true non-nascent geographic atrophy presenting B-scans that are misclassified as nascent geographic atrophy). In cases where the detection controller 710 is deployed for patient screening, those B-scans classified as presenting nascent geographic atrophy may undergo further review and validation. Accordingly, the model may be adjusted to improve recall while maintaining an acceptable number of B-scans classified as nascent geographic atrophy. In an example implementation, the threshold may be increased from a small value with a step size 0.02. The threshold may be chosen such that further increase in the threshold leads to more than 0.2 decrease in recall but only saves less than 1,000 additional B-scans for further review.

In some example embodiments, the detection controller 710 may generate an output for a B-scan exhibiting nascent geographic atrophy that includes one or more dominant bounding box with an above-threshold confidence score. This confidence score may be taken as the confidence score of the B-scan. A successful diagnosis of nascent geographic atrophy B-scan with lesion localization was recorded if and only if the bounding box output overlaps with the ground truth and/or expert annotated bounding boxes.

In some example embodiments, the detection controller 710 may be deployed for AI-assisted diagnosis of nascent geographic atrophy (nGA). In an example embodiment, the detection controller 710 may propose a nascent geographic atrophy diagnosis and one or more lesion locations. For example, the detection controller 710 may identify, within a set of optical coherence tomography volumes, high-risk B-scans for which the underlying deep learning model determines as exhibiting nascent geographic atrophy. These high-risk B-scans may be presented, for example, in a user interface 735 of the client device 730. The absence and presence of nascent geographic atrophy as well as the proposed locations of nascent geographic atrophy lesions may be confirmed based on one or more user inputs received at the client device 730.

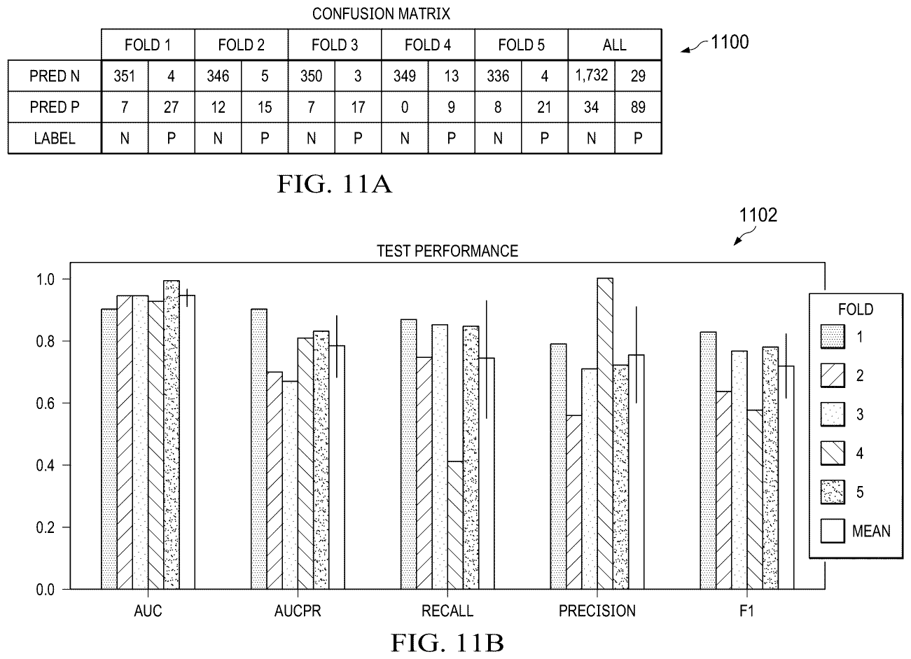

FIG. 11A is an illustration of a confusion matrix 1100 in accordance with one or more example embodiments. The confusion matrix 1100 may be one example of the confusion matrix generated for a 5-fold cross-validation of the performance of a model, such as model 800 in FIGS. 8A-8B. N denotes negative, normal volumes and P denotes positive, nascent geographic atrophy volumes.

FIG. 11B is a graph of statistics for a 5-fold cross-validation in accordance with one or more example embodiments. The area under the curve (AUC), area under the precision-recall curve (AUPRC), recall, precision, and F1-score of the model on the test set from the 5-fold cross-validation are shown in FIG. 11B. The mean performance from the 5 folds is also given, the error bar shows 95% confidence interval (CI). The mean precision and recall are 0.76 (95% CI 0.60˜0.91) and 0.74 (95% CI 0.56˜0.93), respectively.

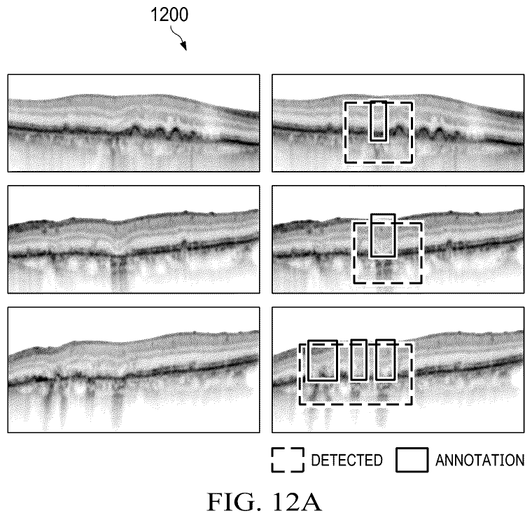

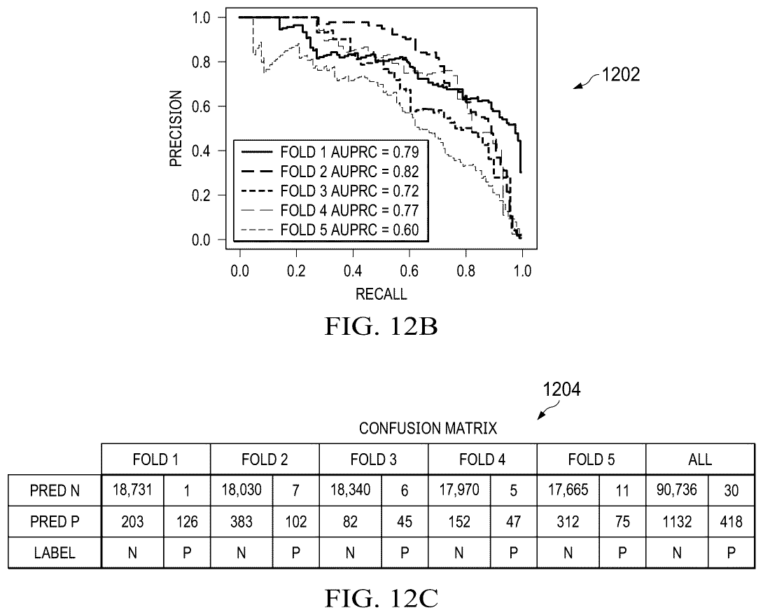

FIG. 12A is an illustration of OCT images 1200 (e.g. B-scans) in which nGA lesions have been detected in accordance with one or more example embodiments. The raw OCT images are shown on the left with the boxes indicating where a human grader annotated for the presence of nGA and where the system (e.g., nascent geographic atrophy detection system 700) identified the presence of nGA). A true positive may be a B-scan where the model-detected bounding box overlaps with expert annotated bounding boxes. A true negative may be a B-scan that includes neither a model-detected bounding box nor annotated bounding box.

FIG. 12B is a graph 1202 of the precision-recall curves for a 5-fold cross validation in accordance with one or more example embodiments.

FIG. 12C is illustration of a confusion matrix 1204 in accordance with one or more example embodiments. In the confusion matrix 1204, N denotes negative B-scan without nascent geographic atrophy (nGA) lesions and P denotes positive B-scans with nascent geographic atrophy (nGA) lesion.

As shown in FIGS. 12A-12C, the detection controller 710 can achieve robust B-scan diagnosis and lesion localization performances without utilizing any B-scan level grading or bounding box annotations. Overall, on the entire example dataset, the recall and precision for B-scan diagnosis with bounding box correctly localized lesions is 0.93 and 0.27, respectively.

Using the type of nascent geographic atrophy detection system 700 described herein may enable more accurate and efficient detection of nGA with a reduction in the amount of time required to process B-scans. As one example, instead of 92,316 individual B-scans, using the nascent geographic atrophy detection system 700, a clinician may need to only review the 1,550 B-scans (or some other number of B-scans) (e.g., about 2%) for which nGA has been detected. Further, using nascent geographic atrophy detection system 700, nGA may be detected where nGA lesions may not have otherwise been detectable by a human grader.

In some example embodiments, the detection controller 710 including the aforementioned deep learning model may be capable of diagnosing nascent geographic atrophy on optical coherence tomography volumes. The detection controller 710 may be capable of performing nascent geographic atrophy diagnosis in a cohort starting with intermediate age-related macular degeneration (iAMD), and no frank geographic atrophy lesion. Nascent geographic atrophy appears to be a significant risk factor for progression to geographic atrophy. The detection controller 710 may be capable of providing diagnosis on individual B-scans and localizing lesions present therein based on optical coherence tomography volume-wise diagnostic labels.

A dataset, such as dataset 725 or training data 900, may be highly unbalanced (e.g., in that a small proportion or 6.26% of cases have nascent geographic atrophy), the use of a B-scan classifier having a pre-trained artificial neural network (ANN) backbone (e.g., a 2D backbone pre-trained on Imagenet data) greatly improves the model performance (F1 score increase from 0.25 to 0.74) for a training dataset of a limited number of OCT volumes.

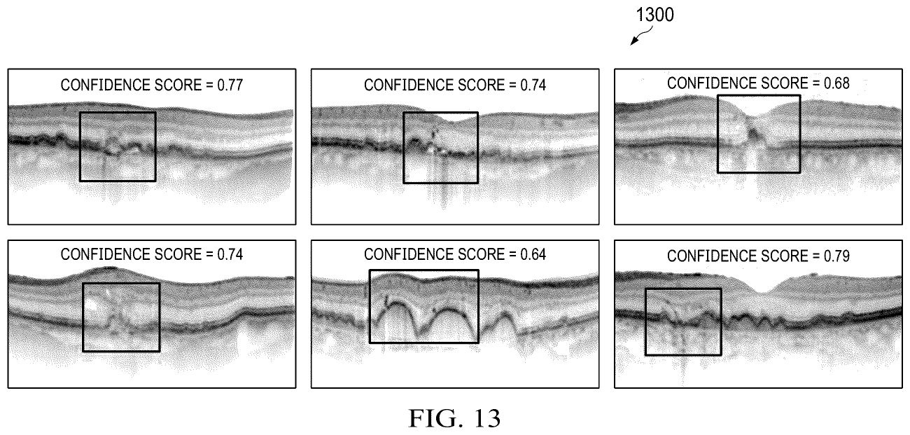

FIG. 13 is an illustration of OCT images 1300 that have been annotated with bounding boxes in accordance with one or more example embodiments. OCT images 1300 show that bounding boxes may be used to locate pathology that presents similarly to nGA (e.g., drusen or hyperreflective foci being connected to the outer plexiform layer (OPL) with retinal pigmented epithelium (RPE); drusen, cyst, or hyperreflective foci creating a subsidence-like structure). Such bounding boxes may be further analyzed by a human grader. In some cases, a higher threshold for the confidence score may be used to exclude pathology other than nGA.

Nevertheless, a weakly supervised method in diagnosing nascent geographic atrophy and localizing nascent geographic atrophy lesions, can assist patient screening if enriching for nascent geographic atrophy, or help in the diagnosis of age-related macular degeneration stage, when using nascent geographic atrophy as a biomarker of progression, or an early endpoint. In clinical trials with nascent geographic atrophy as inclusion/exclusion criterion or clinical biomarkers of progression or endpoints, the grading of nascent geographic atrophy on optical coherence tomography volumes of high density B-scans is laborious and operationally expensive, especially in screening a large population. The proposed AI-assisted diagnosis can greatly alleviate the operational burden and improve the feasibility of such trials. Similar strategies can also be applied to other trials where clinical enrichment is based on multiple anatomical biomarkers.

### V. Exemplary Neural Network

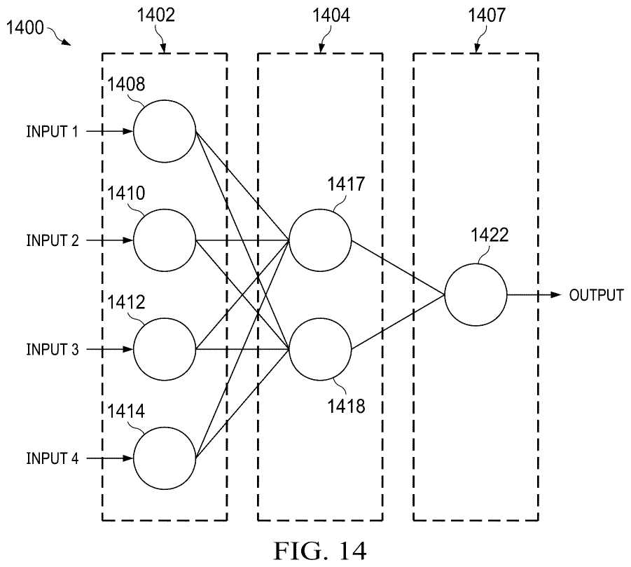

FIG. 14 illustrates an example neural network that can be used to implement a computer-based model according to various embodiments of the present disclosure. For example, the neural network 1400 may be used to implement the model 132 of the health status identification system 101. As shown, the artificial neural network 1400 includes three layers—an input layer 1402, a hidden layer 1404, and an output layer 1407. Each of the layers 1402, 1404, and 1407 may include one or more nodes. For example, the input layer 1402 includes nodes 1408-1414, the hidden layer 1404 includes nodes 1417 and 1418, and the output layer 1407 includes a node 1422. In this example, each node in a layer is connected to every node in an adjacent layer. For example, the node 1408 in the input layer 1402 is connected to both of nodes 1417 and 1418 in the hidden layer 1404. Similarly, the node 1417 in the hidden layer 1404 is connected to all of the nodes 1408-1414 in the input layer 1402 and the node 1422 in the output layer 1407. Although only one hidden layer is shown for the artificial neural network 1400, it has been contemplated that the artificial neural network 1400 used to implement the model 132 may include as many hidden layers as necessary or desired.

In this example, the artificial neural network 1400 receives a set of input values and produces an output value. Each node in the input layer 1402 may correspond to a distinct input value. For example, when the artificial neural network 1400 is used to implement the model 132, each node in the input layer 1402 may correspond to a distinct attribute of an OCT volume image of a retina (e.g., obtained from the OCT imaging system 110 in FIG. 1).

In some embodiments, each of the nodes 1417 and 1418 in the hidden layer 1404 generates a representation, which may include a mathematical computation (or algorithm) that produces a value based on the input values received from the nodes 1408-1414. The mathematical computation may include assigning different weights to each of the data values received from the nodes 1408-1414. The nodes 1417 and 1418 may include different algorithms and/or different weights assigned to the data variables from the nodes 1408-1414 such that each of the nodes 1417 and 1418 may produce a different value based on the same input values received from the nodes 1408-1414. In some embodiments, the weights that are initially assigned to the features (or input values) for each of the nodes 1417 and 1418 may be randomly generated (e.g., using a computer randomizer). The values generated by the nodes 1417 and 1418 may be used by the node 1422 in the output layer 1407 to produce an output value for the artificial neural network 1400. When the artificial neural network 1400 is used to implement the model 132, the output value produced by the artificial neural network 1400 may include a saliency map such as but not limited to a heatmap of the OCT volume image of a retina (e.g., saliency map 144) identifying biomarkers therein.

The artificial neural network 1400 may be trained by using training data. For example, the training data herein may be OCT volume images of retinas. The training data may be, for example, training dataset 148 in FIG. 1. By providing training data to the artificial neural network 1400, the nodes 1417 and 1418 in the hidden layer 1404 may be trained (adjusted) such that an optimal output is produced in the output layer 1407 based on the training data. By continuously providing different sets of training data, and penalizing the artificial neural network 1400 when the output of the artificial neural network 1400 is incorrect (e.g., when incorrectly identifying a biomarker in the OCT volume images), the artificial neural network 1400 (and specifically, the representations of the nodes in the hidden layer 1404) may be trained (adjusted) to improve its performance in data classification. Adjusting the artificial neural network 1400 may include adjusting the weights associated with each node in the hidden layer 1404.

Although the above discussions pertain to an artificial neural network as an example of machine learning, it is understood that other types of machine learning methods may also be suitable to implement the various aspects of the present disclosure. For example, support vector machines (SVMs) may be used to implement machine learning. SVMs are a set of related supervised learning methods used for classification and regression. A SVM training algorithm—which may be a non-probabilistic binary linear classifier—may build a model that predicts whether a new example falls into one category or another. As another example, Bayesian networks may be used to implement machine learning. A Bayesian network is an acyclic probabilistic graphical model that represents a set of random variables and their conditional independence with a directed acyclic graph (DAG). The Bayesian network could present the probabilistic relationship between one variable and another variable. Another example is a machine learning engine that employs a decision tree learning model to conduct the machine learning process. In some instances, decision tree learning models may include classification tree models, as well as regression tree models. In some embodiments, the machine learning engine employs a Gradient Boosting Machine (GBM) model (e.g., XGBoost) as a regression tree model. Other machine learning techniques may be used to implement the machine learning engine, for example via Random Forest or Deep Neural Networks. Other types of machine learning algorithms are not discussed in detail herein for reasons of simplicity and it is understood that the present disclosure is not limited to a particular type of machine learning.

### VI. Example Computing System

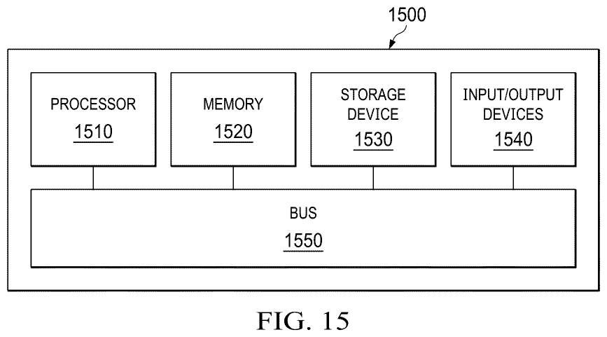

FIG. 15 depicts a block diagram illustrating an example of a computing system 1500, in accordance with some example embodiments. Referring to FIGS. 4, 7-17, and 15, the computing system 1500 may be used to implement the detection controller 710 in FIG. 7, the client device 730 in FIG. 7, and/or any components therein.

As shown in FIG. 15, the computing system 1500 can include a processor 1510, a memory 1520, a storage device 1530, and input/output devices 1540. Computing system 1500 may be one example implementation of health status identification system 101 in FIG. 1. The processor 1510, the memory 1520, the storage device 1530, and the input/output devices 1540 can be interconnected via a system bus 1550. The processor 1510 is capable of processing instructions for execution within the computing system 1500. Such executed instructions can implement one or more components of, for example, the detection controller 710, the client device 730, and/or the like. In some example embodiments, the processor 1510 can be a single-threaded processor. Alternately, the processor 1510 can be a multi-threaded processor. The processor 1510 is capable of processing instructions stored in the memory 1520 and/or on the storage device 1530 to display graphical information for a user interface, such as display system 106 in FIG. 1 or user interface 735 in FIG. 7, provided via the input/output device 1540.

The memory 1520 is a computer readable medium such as volatile or non-volatile that stores information within the computing system 1500. The memory 1520 can store data structures representing configuration object databases, for example. The storage device 1530 is capable of providing persistent storage for the computing system 1500. The storage device 1530 can be a floppy disk device, a hard disk device, an optical disk device, or a tape device, or other suitable persistent storage means. Storage device 1530 may be one example implementation of data storage 104 in FIG. 1. The input/output device 1540 provides input/output operations for the computing system 1500. In some example embodiments, the input/output device 1540 includes a keyboard and/or pointing device. In various implementations, the input/output device 1540 includes a display unit for displaying graphical user interfaces.

According to some example embodiments, the input/output device 1540 can provide input/output operations for a network device. For example, the input/output device 1540 can include Ethernet ports or other networking ports to communicate with one or more wired and/or wireless networks (e.g., a local area network (LAN), a wide area network (WAN), the Internet).

In some example embodiments, the computing system 1500 can be used to execute various interactive computer software applications that can be used for organization, analysis and/or storage of data in various formats. Alternatively, the computing system 1500 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 1540. The user interface can be generated and presented to a user by the computing system 1500 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

### VII. Example Definitions and Context

The disclosure is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion.

Where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

The term “subject” may refer to a subject of a clinical trial, a person or animal undergoing treatment, a person or animal undergoing anti-cancer therapies, a person or animal being monitored for remission or recovery, a person or animal undergoing a preventative health analysis (e.g., due to their medical history), or any other person or patient or animal of interest. In various cases, “subject” and “patient” may be used interchangeably herein.

The term “OCT image” may refer to an image of a tissue, an organ, etc., such as a retina, that is scanned or captured using optical coherence tomography (OCT) imaging technology. The term may refer to one or both of 2D “slice” images and 3D “volume” images. When not explicitly indicated, the term may be understood to include OCT volume images.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, molecular biology, pharmacology and toxicology are described herein are those well-known and commonly used in the art.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

As used herein, the term “about” used with respect to numerical values or parameters or characteristics that can be expressed as numerical values means within ten percent of the numerical values. For example, “about 50” means a value in the range from 45 to 55, inclusive.

The term “ones” means more than one.

As used herein, the term “plurality” can be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, a “model” may include one or more algorithms, one or more mathematical techniques, one or more machine learning (ML) algorithms, or a combination thereof.

As used herein, “machine learning” may include the practice of using algorithms to parse data, learn from it, and then make a determination or prediction about something in the world. Machine learning uses algorithms that can learn from data without relying on rules-based programming.

As used herein, an “artificial neural network” or “neural network” may refer to mathematical algorithms or computational models that mimic an interconnected group of artificial neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in, for example, two ways; when it is being trained (e.g., using a training dataset) it is in training mode and when it puts what it has learned into practice (e.g., using a test dataset) it is in inference (or prediction) mode. Neural networks may learn through a feedback process (e.g., backpropagation) which allows the network to adjust the weight factors (modifying its behavior) of the individual nodes in the intermediate hidden layers so that the output matches the outputs of the training data. In other words, a neural network may learn by being fed training data (learning examples) and eventually learns how to reach the correct output, even when it is presented with a new range or set of inputs.

### VIII. Recitation of Example Embodiments

Embodiment 1: A system, comprising: at least one data processor; and at least one memory storing instructions, which when executed by the at least one data processor, result in operations comprising: applying a machine learning model trained to determine, based at least on an optical coherence tomography (OCT) volume of a patient, a diagnosis of nascent geographic atrophy (nGA) for the patient; determining, based at least on a saliency map associated with the diagnosis of nascent geographic atrophy, a location of one or more nascent geographic atrophy lesions; and verifying the diagnosis of nascent geographic atrophy and/or the location of the one or more nascent geographic atrophy lesions.

Embodiment 2: The system of embodiment 1, wherein the saliency map identifies one or more regions of the optical coherence tomography volume associated with an above-threshold contribution to the diagnosis of nascent geographic atrophy.

Embodiment 3: The system of embodiment 1 or embodiment 2, wherein the saliency map is generated by applying a gradient weighted class activation mapping (GradCAM).

Embodiment 4: The system of any one of embodiments 1-3, wherein the saliency map comprises a heatmap.

Embodiment 5: The system of any one of embodiments 1-4, wherein the machine learning model comprises an artificial neural network (ANN) based classifier.

Embodiment 6: The system of any one of embodiments 1-5, wherein the machine learning model comprises a residual neural network (RNN) based classifier.

Embodiment 7: The system of any one of embodiments 1-6, wherein the optical coherence tomography (OCT) volume comprises a three-dimensional volume having a plurality of two-dimensional B-scans.

Embodiment 8: The system of any one of embodiments 1-7, wherein the machine learning model is trained based on a dataset including a plurality of optical coherence tomography (OCT) volumes annotated with volume-wise labels.

Embodiment 9: The system of any one of embodiments 1-8, wherein the location of the one or more nascent geographic atrophy lesions are identified by one or more bounding boxes.

Embodiment 10: The system of any one of embodiments 1-9, wherein the operations further comprise: generating a user interface displaying an indication of the location of the one or more nascent geographic atrophy lesions on the optical coherence tomography volume of the patient; and verifying, based on one or more user inputs received via the user interface, the diagnosis of nascent geographic atrophy and/or the location of the one or more nascent geographic atrophy lesions.

Embodiment 11: A computer-implemented method, comprising: applying a machine learning model trained to determine, based at least on an optical coherence tomography (OCT) volume of a patient, a diagnosis of nascent geographic atrophy (nGA) for the patient; determining, based at least on a saliency map associated with the diagnosis of nascent geographic atrophy, a location of one or more nascent geographic atrophy lesions; and verifying the diagnosis of nascent geographic atrophy and/or the location of the one or more nascent geographic atrophy lesions.

Embodiment 12: The method of embodiment 11, wherein the saliency map identifies one or more regions of the optical coherence tomography volume associated with an above-threshold contribution to the diagnosis of nascent geographic atrophy.

Embodiment 13: The method of embodiment 11 or embodiment 12, wherein the saliency map is generated by applying a gradient weighted class activation mapping (GradCAM).

Embodiment 14: The method of any one of embodiments 11-13, wherein the saliency map comprises a heatmap.

Embodiment 15: The method of any one of embodiments 11-14, wherein the machine learning model comprises an artificial neural network (ANN) based classifier.

Embodiment 16: The method of any one of embodiments 11-15, wherein the machine learning model comprises a residual neural network (RNN) based classifier.

Embodiment 17: The method of any one of embodiments 11-16, wherein the optical coherence tomography (OCT) volume comprises a three-dimensional volume having a plurality of two-dimensional B-scans.

Embodiment 18: The method of any one of embodiments 11-17, wherein the machine learning model is trained based on a dataset including a plurality of optical coherence tomography (OCT) volumes annotated with volume-wise labels.

Embodiment 19: The method of any one of embodiments 11-18, wherein the location of the one or more nascent geographic atrophy lesions are identified by one or more bounding boxes.

Embodiment 20: The method of any one of embodiments 11-19, wherein the operations further comprise: generating a user interface displaying an indication of the location of the one or more nascent geographic atrophy lesions on the optical coherence tomography volume of the patient; and verifying, based on one or more user inputs received via the user interface, the diagnosis of nascent geographic atrophy and/or the location of the one or more nascent geographic atrophy lesions.

Embodiment 21: A non-transitory computer readable medium storing instructions, which when executed by at least one data processor, result in operations comprising: applying a machine learning model trained to determine, based at least on an optical coherence tomography (OCT) volume of a patient, a diagnosis of nascent geographic atrophy (nGA) for the patient; determining, based at least on a saliency map associated with the diagnosis of nascent geographic atrophy, a location of one or more nascent geographic atrophy lesions; and verifying the diagnosis of nascent geographic atrophy and/or the location of the one or more nascent geographic atrophy lesions.

Embodiment 22. A method comprising: receiving an optical coherence tomography (OCT) volume image of a retina of a subject; generating, via a deep learning model, an output using the OCT volume image in which the output indicates whether nascent geographic atrophy is detected; and generating a map output for the deep learning model using a saliency mapping algorithm, wherein the map output indicates a level of contribution of a set of regions in the OCT volume image to the output generated by the deep learning model.

Embodiment 23. The method of embodiment 22, wherein the saliency mapping algorithm comprises a gradient-weighted class activation mapping (GradCAM) algorithm and wherein the map output visually indicates the level of contribution of the set of regions in the OCT volume image to the output generated by the deep learning model.

Embodiment 24. The method of embodiment 22 or embodiment 23, wherein the OCT volume image comprises a plurality of OCT slice images that are two-dimensional and further comprising: generating an evaluation recommendation based on at least one of the output or the map output, wherein the evaluation recommendation identifies a subset of the plurality of OCT slice images for further review.

Embodiment 25. The method of embodiment 24, wherein the subset includes fewer than 5% of the plurality of OCT slice images.

Embodiment 26. The method of any one of embodiments 22-25, further comprising: displaying the map output, wherein the map output comprises a saliency map overlaid on an individual OCT slice image of the OCT volume image and a bounding box around at least one region of the set of regions.

Embodiment 27. The method of embodiment 26, wherein the identifying comprises: identifying a potential biomarker region in association with a region of the set of regions as being associated with the nascent geographic atrophy; generating a scoring metric for the potential biomarker region; and identifying the biomarker region as including at least one biomarker for the selected diagnosis of nascent geographic atrophy when the scoring metric meets a selected threshold.

Embodiment 28. The method of embodiment 27, wherein the scoring metric comprises at least one of a size of the potential biomarker region or a confidence score for the potential biomarker region.

Embodiment 29. The method of any one of embodiments 22-28, wherein generating the map output comprises: generating a saliency map for an OCT slice image of the OCT volume image using the saliency mapping algorithm, the saliency map indicating a degree of importance of each pixel in the OCT slice image for the diagnosis of nascent geographic atrophy; filtering the saliency map to generate a modified saliency map; and overlaying the modified saliency map on the OCT slice image to generate the map output.

Embodiment 30. The method of any one of embodiments 22-28, wherein generating, via the deep learning model, the output comprises: generating an initial output for each OCT slice image of a plurality of OCT slice images that form the OCT volume image to form a plurality of initial outputs; and averaging the plurality of initial outputs to form the health indication output.

Embodiment 31. A system comprising:
a non-transitory memory; and
a hardware processor coupled with the non-transitory memory and configured to read instructions from the non-transitory memory to cause the system to: receive an optical coherence tomography (OCT) volume image of a retina of a subject; generate, via a deep learning model, an output using the OCT volume image in which the output indicates whether nascent geographic atrophy is detected; generate a map output for the deep learning model using a saliency mapping algorithm, wherein the map output indicates a level of contribution of a set of regions in the OCT volume image to the output generated by the deep learning model; and display the map output.

Embodiment 32. The method of embodiment 31, wherein the map output comprises a saliency map overlaid on an individual OCT slice image of the OCT volume image.

Embodiment 33. The system of embodiment 31 or embodiment 32, wherein the saliency mapping algorithm comprises a gradient-weighted class activation mapping (GradCAM) algorithm.

Embodiment 34. The system of any one of embodiments 31-33, wherein the deep learning model comprises a residual neural network.

Embodiment 35. A system comprising:
a non-transitory memory; and
a hardware processor coupled with the non-transitory memory and configured to read instructions from the non-transitory memory to cause the system to: train a deep learning model using a training dataset that includes training OCT images that have been labeled as evidencing nascent geographic atrophy or not evidencing nascent geographic atrophy to form a trained deep learning model; receive an optical coherence tomography (OCT) volume image of a retina of a subject; generate, via the trained deep learning model, a classification score using the OCT volume image in which the classification score indicates whether nascent geographic atrophy is detected; generate a saliency volume map for the OCT volume image using a saliency mapping algorithm, wherein the saliency volume map indicates a level of contribution of a set of regions in the OCT volume image to the diagnosis of geographic atrophy generated by the deep learning model; detect a set of potential biomarker regions in the OCT volume image using the saliency volume map; and generate a report that confirms that nascent geographic atrophy is detected when at least one potential biomarker region of the set of potential biomarker regions meets a set of criteria and when the classification score meets a threshold.

Embodiment 36. The system of embodiment 35, wherein the saliency mapping algorithm comprises a gradient-weighted class activation mapping (GradCAM) algorithm.

Embodiment 37. The method of embodiment 35 or embodiment 36, wherein the classification score is a probability that the OCT volume image evidences nascent geographic atrophy and wherein the threshold is a value selected between 0.5 and 0.8.

Embodiment 38. The system of any one of embodiments 35-37, wherein the OCT volume image comprises a plurality of OCT slice images that are two-dimensional and wherein the hardware processor is further configured to read instructions from the non-transitory memory to cause the system to generate an evaluation recommendation based on at least one of the health indication output or the map output, wherein the evaluation recommendation identifies a subset of the plurality of OCT slice images for further review, the subset including fewer than 5% of the plurality of OCT slice images.

### IX. Additional Considerations

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

What is claimed is:

1. A method comprising:
receiving an optical coherence tomography (OCT) volume image of a retina of a subject;
generating, via a deep learning model, an output using the OCT volume image in which the output indicates whether nascent geographic atrophy is detected; and
generating a map output for the deep learning model using a saliency mapping algorithm,
wherein the map output indicates a level of contribution of a set of regions in the OCT volume image to the output generated by the deep learning model.

2. The method of claim 1, wherein the saliency mapping algorithm comprises a gradient-weighted class activation mapping (GradCAM) algorithm and wherein the map output visually indicates the level of contribution of the set of regions in the OCT volume image to the output generated by the deep learning model.

3. The method of claim 1, wherein the OCT volume image comprises a plurality of OCT slice images that are two-dimensional and further comprising:
generating an evaluation recommendation based on at least one of the output or the map output, wherein the evaluation recommendation identifies a subset of the plurality of OCT slice images for further review.

4. The method of claim 3, wherein the subset includes fewer than 5% of the plurality of OCT slice images.

5. The method of claim 1, further comprising:
displaying the map output, wherein the map output comprises a saliency map overlaid on an individual OCT slice image of the OCT volume image and a bounding box around at least one region of the set of regions.

6. The method of claim 1, wherein the identifying comprises:
identifying a potential biomarker region in association with a region of the set of regions as being associated with the nascent geographic atrophy;
generating a scoring metric for the potential biomarker region; and
identifying the biomarker region as including at least one biomarker for a selected diagnosis of nascent geographic atrophy when the scoring metric meets a selected threshold.

7. The method of claim 6, wherein the scoring metric comprises at least one of a size of the potential biomarker region or a confidence score for the potential biomarker region.

8. The method of claim 1, wherein generating the map output comprises:
generating a saliency map for an OCT slice image of the OCT volume image using the saliency mapping algorithm, the saliency map indicating a degree of importance of each pixel in the OCT slice image for a diagnosis of nascent geographic atrophy;
filtering the saliency map to generate a modified saliency map; and
overlaying the modified saliency map on the OCT slice image to generate the map output.

9. The method of claim 1, wherein generating, via the deep learning model, the output comprises:
generating an initial output for each OCT slice image of a plurality of OCT slice images that form the OCT volume image to form a plurality of initial outputs; and
averaging the plurality of initial outputs to form a health indication output.

10. A system comprising:
a non-transitory memory; and
a hardware processor coupled with the non-transitory memory and configured to read instructions from the non-transitory memory to cause the system to:
receive an optical coherence tomography (OCT) volume image of a retina of a subject;
generate, via a deep learning model, an output using the OCT volume image in which the output indicates whether nascent geographic atrophy is detected;
generate a map output for the deep learning model using a saliency mapping algorithm,
wherein the map output indicates a level of contribution of a set of regions in the OCT volume image to the output generated by the deep learning model; and
display the map output.

11. The system of claim 10, wherein the saliency mapping algorithm comprises a gradient-weighted class activation mapping (GradCAM) algorithm and wherein the map output visually indicates the level of contribution of the set of regions in the OCT volume image to the output generated by the deep learning model.

12. The system of claim 10, wherein the OCT volume image comprises a plurality of OCT slice images that are two-dimensional and hardware processor is further configured to read instructions from the non-transitory memory to cause the system to:
generate an evaluation recommendation based on at least one of the output or the map output, wherein the evaluation recommendation identifies a subset of the plurality of OCT slice images for further review.

13. The system of claim 12, wherein the subset includes fewer than 5% of the plurality of OCT slice images.

14. The system of claim 10, wherein the map output comprises a saliency map overlaid on an individual OCT slice image of the OCT volume image and a bounding box around at least one region of the set of regions.

15. The system of claim 14, wherein the hardware processor is further configured to read instructions from the non-transitory memory to cause the system to:
identify a potential biomarker region in association with a region of the set of regions as being associated with the nascent geographic atrophy;
generate a scoring metric for the potential biomarker region; and
identify the biomarker region as including at least one biomarker for a selected diagnosis of nascent geographic atrophy when the scoring metric meets a selected threshold.

16. The system of claim 15, wherein the scoring metric comprises at least one of a size of the potential biomarker region or a confidence score for the potential biomarker region.

17. The system of claim 10, wherein the hardware processor is further configured to read instructions from the non-transitory memory to cause the system to:
generate a saliency map for an OCT slice image of the OCT volume image using the saliency mapping algorithm, the saliency map indicating a degree of importance of each pixel in the OCT slice image for a diagnosis of nascent geographic atrophy;
filter the saliency map to generate a modified saliency map; and
overlay the modified saliency map on the OCT slice image to generate the map output.

18. The system of claim 10, wherein the map output comprises a saliency map overlaid on an individual OCT slice image of the OCT volume image.

19. A system comprising:
a non-transitory memory; and
a hardware processor coupled with the non-transitory memory and configured to read instructions from the non-transitory memory to cause the system to:
train a deep learning model using a training dataset that includes training OCT images that have been labeled as evidencing nascent geographic atrophy or not evidencing nascent geographic atrophy to form a trained deep learning model;
receive an optical coherence tomography (OCT) volume image of a retina of a subject;
generate, via the trained deep learning model, a classification score using the OCT volume image in which the classification score indicates whether nascent geographic atrophy is detected;
generate a saliency volume map for the OCT volume image using a saliency mapping algorithm, wherein the saliency volume map indicates a level of contribution of a set of regions in the OCT volume image to a diagnosis of geographic atrophy generated by the deep learning model;
detect a set of potential biomarker regions in the OCT volume image using the saliency volume map; and
generate a report that confirms that nascent geographic atrophy is detected when at least one potential biomarker region of the set of potential biomarker regions meets a set of criteria and when the classification score meets a threshold.

20. The system of claim 19, wherein the saliency mapping algorithm comprises a gradient-weighted class activation mapping (GradCAM) algorithm and wherein the classification score is a probability that the OCT volume image evidences nascent geographic atrophy and wherein the threshold is a value selected between 0.5 and 0.8.
